(19) United States
(12) Patent Application Publication
(10) Pub. No.: US 2013/0123583 A1
(43) Pub. Date: May 16, 2013

(54) SYSTEM AND METHOD FOR ANALYZING DIGITAL MEDIA PREFERENCES TO GENERATE A PERSONALITY PROFILE

(71) Applicant: Erica L. Hill, Fort Lauderdale, FL (US)

(72) Inventor: Erica L. Hill, Fort Lauderdale, FL (US)

(21) Appl. No.: 13/675,799

(22) Filed: Nov. 13, 2012

Related U.S. Application Data
(60) Provisional application No. 61/558,233, filed on Nov. 10, 2011.

(51) Int. Cl. G06F 17/30; A61B 5/00

(52) U.S. Cl. CPC G06F 17/30595; A61B 5/00
USPC 600/300; 434/236; 707/736

(57) ABSTRACT

A system and method for assessing a personality profile includes a database that holds known control data that includes correlations between musical preference patterns and psychological indications. The system also includes a processor that executes an input module, a processing module, and an output module. The input module receives information relating to a history of media selections of an assessment subject. The input module also receives a response to a questionnaire that includes data relating to the musical preference of the assessment subject. The processing module analyzes the information relating to the history of media selections and the response to the questionnaire by comparing the musical preference to the musical preference patterns in the database. The output module provides a personality profile of the assessment subject based on the analysis.

### RELATED APPLICATIONS

This application claims the benefit of U.S. Provisional Patent Application No. 61/558,233 entitled “System and Method for Analyzing the Content of Musical Preferences in Order to Achieve a Psychological Profile” filed Nov. 11, 2011, the entire contents of which are incorporated herein by reference.

### FIELD OF THE INVENTION

The present invention relates to systems and methods for creating a personality profile. More specifically, the invention relates to systems and methods for analyzing the content of media preferences of an individual in order to achieve a personality profile.

### BACKGROUND OF THE INVENTION

Mental health disorders are the most common type of disability for young adults. Such disorders typically require significant expenditure of health care and financial resources. In fact, mental health disorders among young adults account for approximately fifty-percent of the world's disease burden, U.S. Surgeon General reports indicate that today's teens and young adults are at particularly high risk for experiencing some kind of mental health related illness.

Proper diagnosis of mental health disorders is key to successful treatment. A number of different psychological screening tools and personality assessment methods are typically applied for diagnosis of teens and young adults. Two well-known personality assessment tools are the Minnesota Multiphasic Personality Inventory (MMPI) and The Rorschach Inkblot Test.

The MMPI was originally developed in the late 1930s, and is one of the personality tests most frequently used by mental health professionals to assess and diagnose mental illness. The first major revision to the MMPI, commonly referred to as the MMPI-2, is appropriate only for adults. The test currently comprises a questionnaire of 567 evaluation items, all true-or-false in format. Administration of the test usually takes between one to two hours depending on the reading level of the individual being evaluated. MMPI-A is a tailored version of the MMPI-2 that is used for adolescents.

The Rorschach Inkblot Test is a psychological test in which an individual's perceptions of inkblots are recorded and then analyzed using psychological interpretation, complex algorithms, or both. Psychologists may use the Rorschach Inkblot Test to examine an individual's personality characteristics and emotional functioning. Inkblot interpretation is often employed in cases where patients are reluctant to describe their thinking processes openly.

Successful application of both of the psychological assessment methods described above depends upon an individual's consent and participation, motivation, insight, and communication skills. Both methods also presume the test subject's ability to access and pay for services, either directly or via some form of health insurance. However, teens and young adults are not typically inclined to admit they may have a mental health problem, or to seek out psychological services even if they do realize and admit they have a problem. Others may not have the financial means to pay for diagnostic services. Consequently, many young people suffering from mental health related issues, illnesses, and disorders remain unidentified and untreated.

Unfortunately, research into less obtrusive and more affordable psychological assessment methods and tools has been limited. A largely unexplored approach to overcoming the resistance to communicating that is typical of the 15 to 30 year old age group (currently known as “Generation Y”) is exploiting this group's receptiveness both to technology and to music.

Generation Y is a demographic known to be technology savvy and to be heavy consumers of technological gadgetry. Behavioral assessment techniques that incorporate technology present an opportunity to engage young people in a less officious manner. Advancements have been made in the area of technology-based evaluation of children, teens and young adults based either upon data obtained from existing psychological measures or upon data newly derived from a variety or possible procedures and methods. But contemporary methods still require the child, teen or young adult to individually meet with an examiner and engage in some degree of interaction. As is the case with traditional diagnostic approaches, such individualized assessment and identification of individuals in need is time consuming and costly.

Analysis of music preferences presents another opportunity for personality assessment of young people. Revealed preference monitoring has been used to target advertising and product placement to young people in a minimally obtrusive manner. But, mental health diagnosis that uses music as a medium and that applies principles from well-known assessments such as MMPI-2 and Rorschach remains a largely unexplored focus area in the art.

U.S. Pat. No. 5,848,396 to Gerace discloses analyzing computer activity and viewing habits of an end user to form a psychographic profile. The profile may be used, along with additional user demographics, to auto-target and customize advertisements to selected users. U.S. Patent Publication No. 2012/0059785 by Pasqual Leo et al. and U.S. Pat. No. 8,131,271 to Ramer et al. both disclose monitoring a website and/or mobile telephone for downloads, and calculating user personality profiles for the purpose of adapting content to individual users. Although both of the implementations above attempt to generate a user personality profile, neither focuses on revealed preference data related specifically to music downloads. Furthermore, neither implementation includes rules for diagnosis of mental health issues, illness, and disorders, nor of the predisposition to such.

U.S. Patent Publication No. 2006/0161553 by Woo et al. discloses monitoring of user interactions with a network and analyzing content from those interactions using psychological dimensions. The implementation may include a linguistic analysis component that may score content segments based on linguistic parameters. However, the disclosed implementation does not analyze musical parameters, nor augment the analysis with questionnaire data tailored to further characterize the automatically monitored user interactions.

U.S. Patent Publication No. 2006/0005226 by Lee discloses development of a user profile based initially on solicited input (such as by questionnaire) regarding content of interest to a user, and based subsequently on media and content actually downloaded by the user. The user profile may then be used to synchronize content on the user's player and to automatically generate new download lists. However, the disclosed implementation does not analyze user input by comparison with correlations between download preference patterns and psychological indications.

Accordingly, there is a need for improved systems and methods for evaluating the mental health of teens and young adults that do not require direct and/or individualized examination or interaction. Furthermore, there is a need for improved mental health evaluation systems and methods that analyze revealed preferences made by the individual for purposes other than a psychological evaluation. Also, there exists a need in the industry for improved systems and methods that generate a mental health evaluation and personality profile by unobtrusively exploiting an individual's routine technology usage and revealed music preferences.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the foregoing in mind, embodiments of the present invention are related to systems and methods for analyzing content of media preferences of an individual in order to achieve psychological data about that individual. An individual's history of media preferences (e.g., music playlist), along with that individual's responses to a questionnaire that may be filled out in conjunction with a submitted music preference history, may be analyzed by comparison against a known database. A method for scoring analysis results may be used to generate a personality profile that may predict, profile, and evaluate individuals.

The present invention advantageously may provide a system and method for analyzing the content of media or musical preferences of an individual to achieve a personality profile. This advantageously readily allows a mental health professional to facilitate understanding an individual's overall mental health and wellbeing. This also advantageously allows for ready identification any potential causes for concern or predisposition to mental health issues, illness or disorders. It is further an object of the present invention to advantageously appeal to the mode of communicating (technology and music) that may target an at-risk age group, and may be able to be administered unobtrusively in any setting that is common and convenient to that age group. More specifically, the system and method of the invention advantageously may require minimal active participation on the part of the individual being evaluated.

It is further an object of the present invention to advantageously provide information that may be useful for a wide variety of applications including, but not limited to, mental health diagnostics, career and academic counseling, social networking, vocational screening, military and government evaluations, and data raining. It is further an object of the present invention to leverage the target group's knowledge and comfort with technology to reveal information in an objective way, including information not understood, appreciated, or even known by the individuals themselves. It is still further an object of the present invention that a range of media, including music, apps, computer data and television, advantageously may be individually or collectively analyzed and aggregated to facilitate a complete, overall, and detailed personality profile that may be helpful as a mental health diagnostic tool.

These and other objects, features and advantages according to embodiments of the present invention are provided by personality profile assessment systems and methods that may feature musical preference information. The present invention may comprise a database configured to hold known control data that includes correlations between musical preference patterns and psychological indications. The present invention may also include a processor configured to execute an input module, a processing module, and an output module.

More particularly, the present invention may include an input module that may receive information relating to a history of media selections by an assessment subject. The history of media selections may include musical preferences, and may be stored in a memory and accessible using a user interface. The user interface may be configured to execute on a smart phone, a portable music player, a portable computer, and/or a digital video recorder. The information relating to musical preference of an assessment subject may be in the form of a music playlist selection, a downloaded apps selection, a browser history selection, a downloaded images selection, a downloaded videos selection, a television recordings selection, and/or a browser cookie selection. The input module also may receive a response to a questionnaire that may include data relating to musical preferences of the assessment subject. The questionnaire and/or the history of media selections may be trans table over a network.

The present invention may have a processing module that may analyze the information relating to the history of media selections and the response to the questionnaire. More particularly, the processing module may compare a musical preference of the assessment subject to musical preference patterns in the control data. The processing module may assign a questionnaire scale score and/or a total questionnaire score to the questionnaire response based on a media preference, a media selection process, a demographic factor, and/or a functioning history factor. The processing module may assign a playlist scale score and an overall playlist score to the music playlist selection based on thematic content, lyrical content, artist characteristics, and/or musical content. The processing module also may determine an aggregate scale score and an overall aggregate score.

The present invention may include an output module that may provide a personality profile of the assessment subject based on an analysis completed by the processing module. More particularly, the output module may generate a personality profile and/or a mental health diagnosis. The personality profile may include a total score and/or a personality profile scale. The mental health diagnosis may include a finding of psychological indications comprising a mental health issue, a mental illness, a mental disorder, and/or a mental health predisposition.

A method aspect of the present invention may be for assessing personality and profiling which may include the steps of receiving information relating to a history of media selections, receiving a response to a questionnaire, analyzing the information relating to the history of media selections and the response to the questionnaire, and providing a personality profile of the assessment subject based on the analysis.

More particularly, the history of media selections may include a musical preference of an assessment subject, and may be stored in a memory and accessible using a user interface. The questionnaire response may include data relating to the musical preference of the assessment subject. The computer-implemented interface may execute on a device selected such as, for example, a smart phone, a portable music player, a portable computer, or a digital video recorder. Both the questionnaire and the history may be transmittable over a network.

Analyzing the information relating to the history of media selections may further include comparing the musical preference to the musical preference patterns that may be held in a database of known control data that may include correlations between musical preference patterns and psychological indications. The musical preference may comprise a music playlist selection, a downloaded apps selection, a browser history selection, a downloaded images selection, a downloaded videos selection, a television recordings selection, and/or a browser cookie selection. In cases where the musical preference includes a music playlist selection, the method step of analyzing the information may include assigning a score to the music playlist selection based on one or more of thematic content, lyrical content, artist characteristics, and musical content.

Analyzing the information may also include assigning a score to the questionnaire response based on a media preference, a media selection process, a demographic factor, and/or a functioning history factor. The method step of assigning the score to the music playlist selection may include determining a playlist scale score and an overall playlist score, an aggregate scale score, and/or an overall aggregate score. Assigning the score to the questionnaire response may further comprise determining a questionnaire scale score and a total questionnaire score.

The method step of providing a personality profile of the assessment subject may comprise generating a personality profile and/or a mental health diagnosis. The personality profile may include a total score and/or a personality profile scale. The mental health diagnosis may include a finding of psychological indications comprising at least one of a mental health issue, a mental illness, a mental disorder, and/or a mental health predisposition.

### BRIEF DESCRIPTION OF THE DRAWINGS

FIG. 1 is a schematic organizational diagram of a computes-based system for analyzing digital media preferences to generate a personality profile according to an embodiment of the present invention.

FIG. 2 is a flowchart illustrating a method aspect of an embodiment of the present invention for analyzing digital media preferences to generate a personality profile according to an embodiment of the present invention.

FIG. 3 is a diagram Illustrating a screen shot of a media selection history for use in connection with the system illustrated in FIG. 1 according to an embodiment of the present invention.

FIGS. 4a to 4c are diagrams illustrating a playlist questionnaire for use in connection with the system illustrated in FIG. 1 according to an embodiment of the present invention.

FIG. 5 is a flowchart illustrating a method aspect according to an embodiment of the present invention for performing validity screening as illustrated in FIG. 2.

FIG. 6 is a flowchart illustrating a method aspect according to an embodiment of the present invention for categorizing an assessment subject as illustrated in FIG. 2.

FIG. 7 is a flowchart illustrating a method aspect according to an embodiment of the present invention for analyzing special scores as illustrated in FIG. 5.

FIGS. 8a to 8c are diagrams illustrating a playlist questionnaire scoring templates for use in connection with the system illustrated in FIG. 1 according to an embodiment of the present invention.

FIG. 9 is a flowchart illustrating a method aspect according to an embodiment of the present invention for analyzing critical items as illustrated in FIG. 5.

FIG. 10 is a flowchart illustrating a method of analyzing music variables of a song according to an embodiment of the present invention for performing playlist scoring as illustrated in FIG. 2.

FIGS. 11a and 11b are flowcharts illustrating a method of analyzing artist remarkable feature variables of a song according to an embodiment of the present invention for performing playlist scoring as illustrated in FIG. 2.

FIG. 12 is a flowchart illustrating a method of analyzing distinction variables according to an embodiment of the present invention for processing distinctions as illustrated in FIG. 11b.

FIG. 13 is a flowchart illustrating a method of analyzing album information variables according to an embodiment of the present invention for performing playlist scoring as illustrated in FIG. 2.

FIG. 14 is a flowchart illustrating a method of analyzing lyrical theme variables according to an embodiment of the present invention for performing playlist scoring as illustrated in FIG. 2.

FIG. 15 is a flowchart illustrating a method of analyzing repeated phrase variables according to an embodiment of the present invention for performing playlist scoring as illustrated in FIG. 2.

FIG. 16 is a flowchart illustrating a method of analyzing word count variables according to an embodiment of the present invention for performing playlist scoring as illustrated in FIG. 2.

FIG. 17 is a diagram illustrating a screen shot of a media selection history for use in connection with the system illustrated in FIG. 1.

FIG. 18 is a diagram illustrating another screen shot of a media selection history for use in connection with the system illustrated in FIG. 1.

FIG. 19 is a diagram illustrating a screen shot of a personality profile for use in connection with the system illustrated in FIG. 1.

FIG. 20 is a flowchart illustrating a method of delivering a diagnosis according to an embodiment of the present invention for constructing a personality profile as illustrated in FIG. 2.

FIG. 21 is a flowchart illustrating a method of delivering another diagnosis according to an embodiment of the present invention for constructing a personality profile as illustrated in FIG. 2.

FIG. 22 is a block diagram illustrating a diagrammatic representation of a machine in the example form of a computer system according to an embodiment of the present invention.

### DETAILED DESCRIPTION OF THE PREFERRED EMBODIMENT

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The present disclosure relates to a system and method for analyzing musical preference patterns to generate a personality profile of an individual. However, it is understood that the following disclosure provides many different embodiments or examples. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

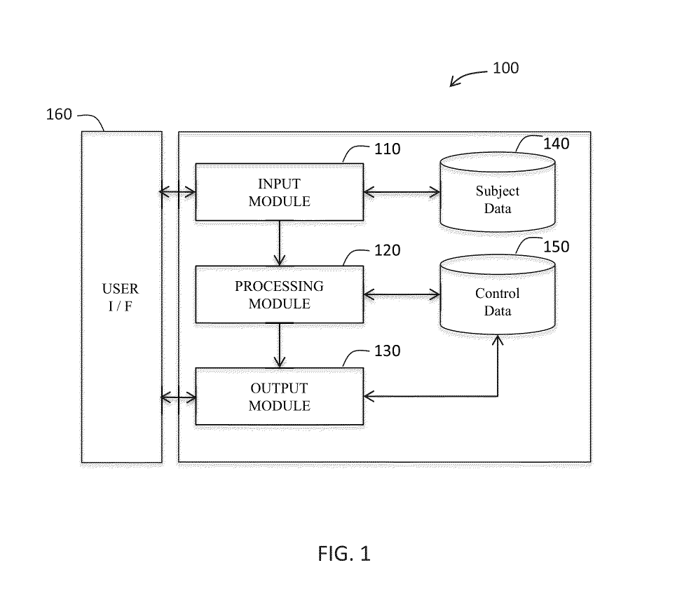

Referring now to FIGS. 1-22, a system 100 for analyzing digital media preferences to generate a personality profile is now described in greater detail. Throughout this disclosure, the present invention may be referred to as a personality profiling system 100, a computer program product, a computer program, a product, a system, a tool, and a method. Furthermore, the present invention may be referred to as relating to music playlists, playlists, streaming playlists, music downloads, and music videos. Those skilled in the art will appreciate that this terminology is only illustrative and does not affect the scope of the invention as outlined herein. For instance, the present invention may just as easily relate to video files, webpage content, digital files, or other digital representations.

### System Architecture

FIG. 1 illustrates a block diagram of the architecture of the system 100 for analyzing digital media preferences to generate a personality profile in accordance with an embodiment of the present invention. The personality profiling system 100 may include an input module 110, a processing module 120, an output module 130, and one or more databases 140, 150. A user of the personality profiling system 100 may interact with the input 110 and output 130 modules via a user interface 160. The input module 110 may be used to receive revealed preference data (e.g., musical preference patterns) related to an individual, and may interact with a database 140 to store such subject data. The processing module 120 may be used to analyze the revealed preference data as compared to psychological indications stored in a database 150 as control data. The output module 130 may be used to correlate analysis results from the processing module with diagnosis rules stored as control data to provide a personality profile to a user through the user interface 160. Although illustrated as including a pair of databases 140, 150, those skilled in the art will appreciate that the system 100 according to an embodiment of the present invention may include any number of databases stored on a single or multiple storage devices. The input module 110, processing module 120, output module 130, databases 140, 150 and user interface 160 will be described individually in greater detail below in the context of the method steps each system component may be configured to carry out.

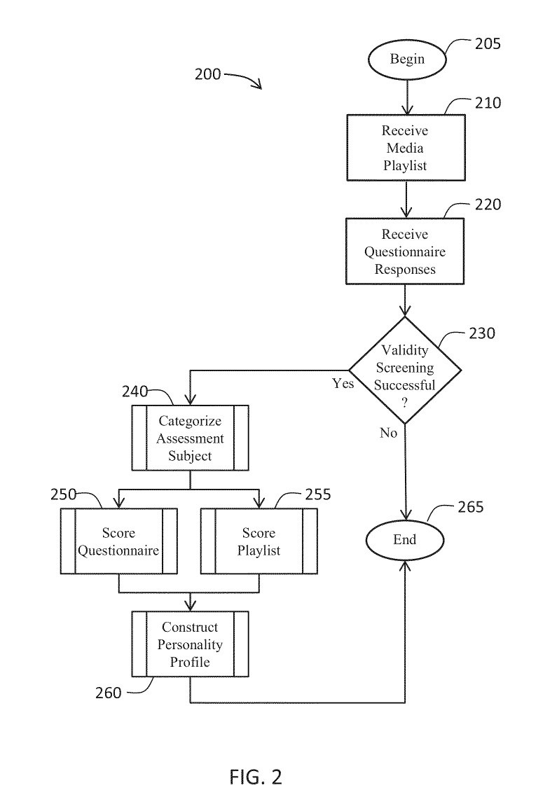

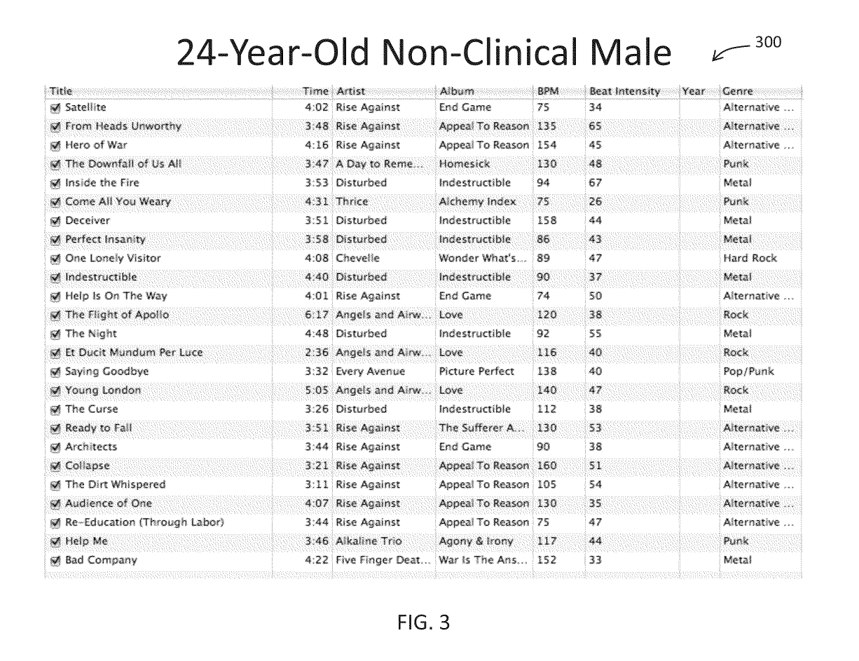

Referring now additionally to the flowchart 200 of FIG. 2, the general operation of the system 100 according to an embodiment of the present invention is now described. From the start (Block 205), a media playlist may be received at Block 210. The media playlist may be received by the input module 110 and may include information relating to a history of media selections made by an individual. For example, and without limitation, the history of media selections may be in the form of a music playlist, an example of which is illustrated as 300 in FIG. 3, which will be described in greater detail below. These media selections may be stored in a database 140 for subsequent retrieval.

The method may continue with receipt by the input module 110 of responses to a questionnaire (Block 220). The responses may relate, at least in part, to the history of media selections from Block 210.

At Block 230, a validity screening is performed with respect to the media selections. More particularly, the history of media selections may be screened by the input module 110 to ascertain whether the evaluation method is applicable to the individual and whether the input sample size supports application of the remaining steps of the method.

If it is determined at Block 230 that the validity screening is successful, then at Block 240, the assessment subject is categorized. More specifically, pre-processing of some subset of the responses to the questionnaire and/or entries in the playlist may by employed to categorize the individual being assessed. If, however, it is determined at Block 230 that the validity screening is not successful, then the method is ended at Block 265.

The responses to the questionnaire are scored at Block 250, and the entries in the playlist are scored at Block 255. The completed questionnaire and the individual's media selection history may be stored in a database 150 for subsequent retrieval, and may be analyzed using the processing module 120 to assess correlations between those scores and psychological indications.

Thereafter, a personality profile may be constructed at Block 260. More particularly, the output module 130 may generate the personality profile based on analysis of results from Blocks 250 and 255, before the method terminates (Block 265).

The method steps for receipt of a media playlist 210, receipt of questionnaire responses 220, performance of validity screening 230, categorization of the assessment subject 240, scoring of the questionnaire 250 and playlist 255, and generation of a personality profile 260 will be described individually in greater detail below.

### Content: Playlist

The content of the history of media selections received (Block 210) by the input module 110 is now described in more detail. A typical media selection history may include a list of music selections made by an individual during the normal course of that individual's interaction with a music delivery device. The playlist may be created in any computer audio program, and may be based upon a frequency count of the individual's most listened to songs as logged in the library of the computer audio program. More specifically, the playlist may be used as input to an analysis of an individual's downloaded applications and/or pattern of downloading applications to a music delivery device. For example, the music delivery device may include, but is not limited to, a computer, iPhone, iPod, Android-enabled device, MP3 player, or similar computerized and/or portable device.

Referring now additionally to FIG. 3, a screenshot of a playlist 300 is depicted. An individual may submit a playlist 300 of their “Top 25 Most Frequently Listened To Playlist.” For example, and without limitation, this list may be automatically generated in a commercially available application such as iTunes. The individual being evaluated also may provide access to personal musical playlists on one or more music delivery devices to facilitate scoring (as described in the above flowchart 200 with reference to Block 255) and profiling (as described in the above flowchart 200 with reference to Block 260) of the test subject using the system 100 according to an embodiment of the present invention. In the history of media selections received (as described in the above flowchart 200 with reference to Block 210) may include an individual's web browser history, which may include (1) items shown in cache, (2) websites that have been visited, (3) downloaded images or videos, and/or (4) browser cookies. The media selections history may, in addition to or alternately, list television shows recorded and scheduled to be recorded on a device such as a digital video recorder.

### Content: Questionnaire

Referring now back to the flowchart 200 in FIG. 2, questionnaire responses from an individual may be received at Block 220 by the input module 110. The questionnaire responses may include test subject answers to questions that may be specifically developed to accompany playlist submissions. Along with demographic information, this questionnaire may capture information about the input playlist and the individual's listening preferences. Additional information regarding an individual's history and functioning may also be obtained. For example, and without limitation, a questionnaire may comprise query items that may be grouped into two primary focus areas: Listening Preferences and Clinical Questions.

The listening preferences query items may be designed to elicit information pertaining to an individual's music choices, selection processes, and download practices. The listening preferences query items may be implemented as rules within the system 100. The rules may be stored as control data in a database 150, and may establish a normative basis for a comparison group as applied to mental health assessment. For example, and without limitation, the rules may be developed by gathering information related to digital media players, listening patterns, and music downloads obtained from pilot questionnaires given to young people between the ages of 15 and 30 years old.

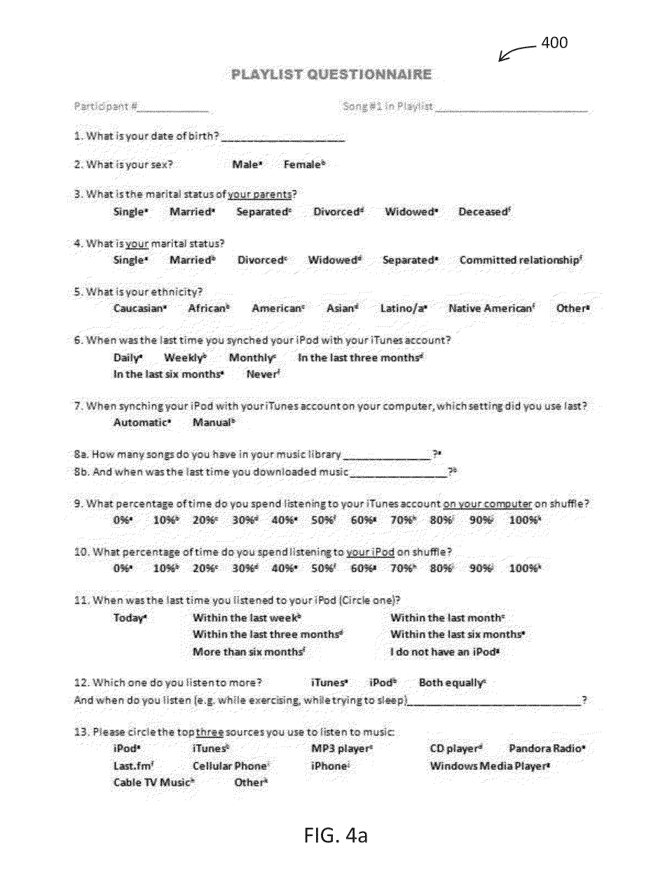

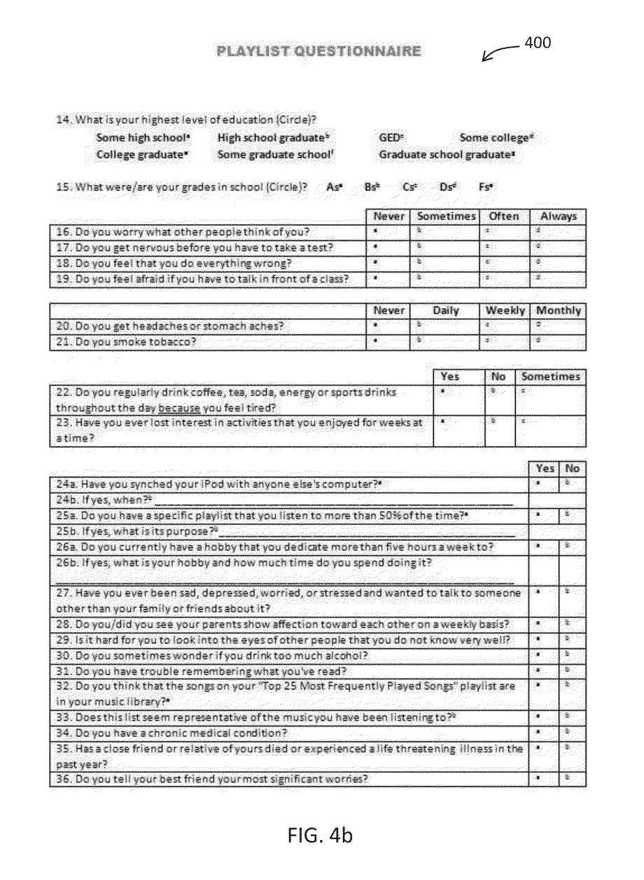

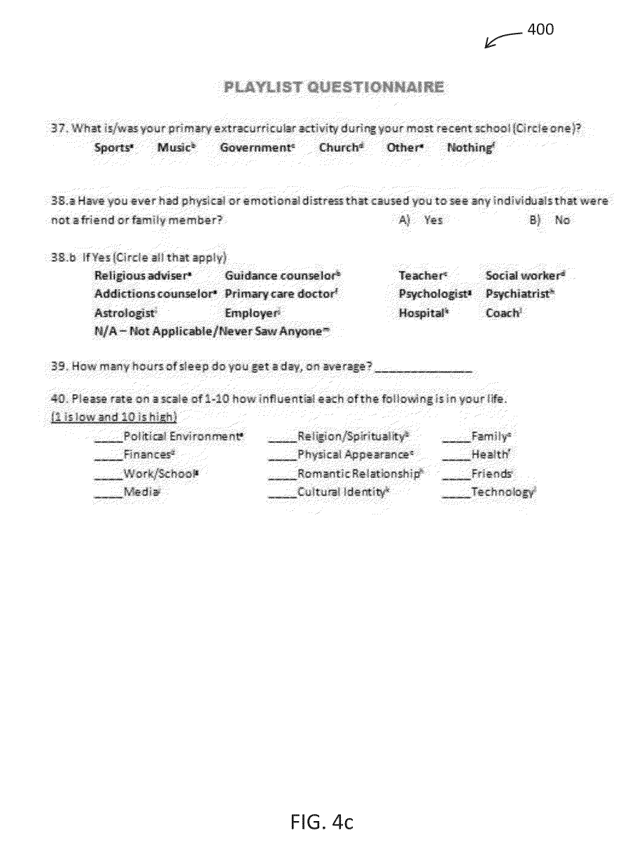

FIGS. 4a and 4b illustrate screenshots of portions of a sample questionnaire 400 containing typical listening preference query items. For example, and without limitation, listening preference query items may include the following:

1. How many songs do you have in your music library?

2. When was the last time you downloaded music?

3. Do you have a specific playlist that you listen to more than 50% of the time?

4. What percentage of time do you spend listening to your iTunes account with your computer on shuffle?

The clinical questions query items may ascertain various details related to the test subject's demographics, health concerns, social history, and work and occupational functioning. The clinical questions query items may be implemented as rules within the system 100. The rules may be stored as subject data in a database 140, and may be modeled after questions typically used in a diagnostic clinical interview, as well as in existing personality assessments and screening tools.

FIGS. 4a, 4b, and 4c illustrate sample clinical question query items that may be present in a typical questionnaire 400. For example, and without limitation, clinical questions query items may include the following:

1. Do you tell your best friend your most significant worries?

2. What were/are your grades in school?

3. Do you ever wonder if you drink too much alcohol?

4. Is it hard for you to look into the eyes of other people that you do not know very well?

5. Have you ever been sad, depressed, worried, or stressed and wanted to talk to someone other than your family or friends about it?

### Validity Screening

Validity problems commonly associated with self-reporting measures such as questionnaires may include social desirability bias and fear of reprisal bias, and must be accounted for in a psychological analysis. More specifically, threats to validity in personality assessment may be viewed as coming from two sources: intrapersonal factors and interpersonal factors. Intrapersonal factors are those inherent to the individual (e.g., internal mental processing issues such as memory recall, verbal skills, and comprehension). Interpersonal factors are those outside of the individual (e.g., external factors in the environment and/or dynamics between the individual and others which interfere with the validity of the results). For example, if an individual change responses to appear more favorable or if concerns about confidentiality of privacy alter the individual's responses, the results may be invalidated.

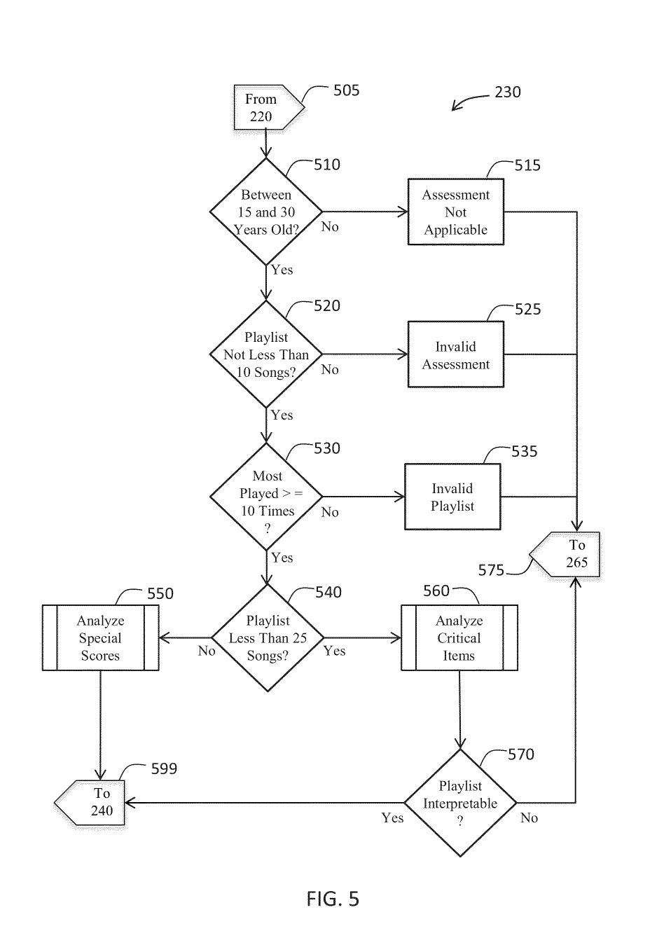

Referring now to the flowchart 230 illustrated in FIG. 5, the step of pre-assessment validity screening is described in greater detail. More specifically, the flowchart 230 illustrated in FIG. 5 merely provides enhanced detail of the step of performing validity screening (Block 230 in the flowchart 200 of FIG. 2) and, as such, is similarly labeled. The step of performing validity screening may be accomplished to determine if the media selection history data received by the input module 110 supports a valid assessment using the system 100 according to an embodiment of the present invention. The playlist provided by the test subject may be referenced to query validity factor rules enforced by the system 100. From the start (Block 505), the test subject's birth date may be checked at Block 510 to confirm inclusion in the target group (for example, and without limitation, 15- to 30-years old) for which the assessment of the present invention 100 may be tailored. If the test subject is not of the targeted age, the assessment is not applicable (Block 515) and the method may end (Block 575). If, however, the assessment is appropriate for the age group of the test subject (Block 510), then the playlist may be checked for validity.

More specifically, at Block 520, it may be determined whether the playlist length is suitable. If it is determined at Block 520 that the playlist length is not suitable, then the playlist may be deemed invalid and insufficient to support assessment (Block 525), and the method is ended at Block 575. If, however, it is determined at Block 520 that the playlist length is valid, then it is determined at block 530 whether or not the playlist is sufficiently played. As illustrated in the flowchart 230 of FIG. 5, the preferred playlist length is less than ten media entries (Block 520), but those skilled in the art will appreciate that any number of media entries may be used to determine whether or not the playlist length is sufficient.

At Block 530, it is determined whether or not the most frequently played song on the playlist is played a sufficient number of times to support assessment. For exemplary purposes, the threshold play count illustrated in the flowchart 230 of FIG. 5 is less than ten plays (Block 530), but those skilled in the art will appreciate that the play count may be set at any number that is desired. If it is determined at Block 530 that the threshold play count has not been met or exceeded, then the playlist may be determined to be invalid at Block 535, and the method is ended at Block 575. If, however, the playlist is determined to be valid at Block 530, then it is determined at Block 540 whether or not the number of songs in the playlist is of sufficient size to use special scores from the playlist to support pre-assessment categorization of the test subject, or if critical items from the questionnaire must be used to support categorization. In the example listed in the flowchart 230 of FIG. 5, the threshold number of songs in the playlist for supporting this determination at Block 540 is 25. If the number of songs in the playlist meets the threshold set at Block 540, then analysis of special scores from the playlist (Block 550) may drive categorization of the assessment subject at Block 599 (Block 230 in the flowchart 200 of FIG. 2). If the number of songs in the playlist does not meet the threshold set at Block 540, then analysis of critical items from the questionnaire, which may containing questions relating at least in part to the playlist, may be analyzed to determine if the playlist content received by the input module 110 is valid and interpretable (Block 570) by the processing module 120 despite the small sample size of the playlist. If it is determined at Block 570 that the playlist cannot be interpreted, then the method is ended at Block 570. If it is determined at Block 570 that the playlist can be interpreted, then analysis of critical items from the questionnaire (Block 560) may drive categorization of the assessment subject at Block 599 (Block 230 in the flowchart 200 of FIG. 2).

### Subject Categorization

Characterizing this assessment subject may be based on analysis of critical items that may characterize whether an individual's responses and mental health history characterize the assessment subject as belonging to a group defined as clinical, non-clinical, or some combination of clinical and non-clinical. For the purposes of this disclosure, a clinical group may be defined as anyone who meets the following criteria: 1) the subject has received a psychological diagnosis by a professional in the past or present, 2) the subject reports having talked with someone about psychological problems in the past or present, and/or 3) the subject reports desiring to talk to someone other than a family member or friend in the past or present related to feelings of “sadness, depression, worry, or stress”.

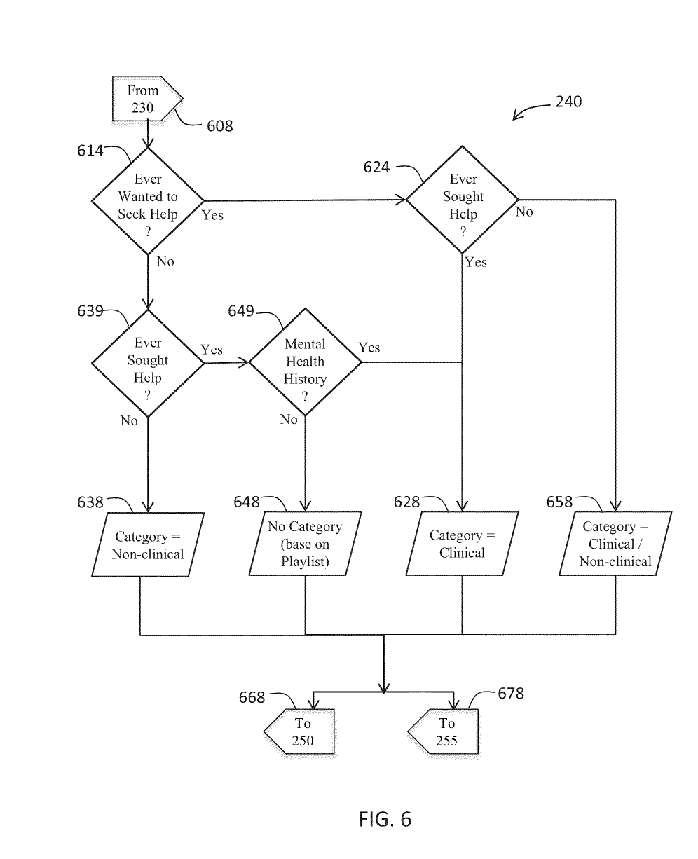

Referring now to the flowchart 240 illustrated in FIG. 6 (which describes further details of Block 240 in the flowchart 200 illustrated in FIG. 2), categorization of the assessment subject is now discussed in greater detail.

From the start (Block 608), it is determined whether or not the test subject ever wanted to seek help for emotional issues at Block 614. If it is determined at Block 614 that the test subject did ever want to seek help, then it is determined at Block 624 whether or not the test subject ever did seek help. If it is determined at block 624 that the test subject has sought help, then the test subject may be categorized as clinical at Block 628. If, however, it is determined at Block 624 that the test subject did not seek help in the past, then the test subject may be categorized as clinical/nonclinical at Block 658, and the method continues at Blocks 668 and 678 with scoring (more specifically, scoring of the questionnaire at Block 250 and scoring of the playlist at Block 255, both from the flowchart 200 illustrated in FIG. 2).

Continuing to refer to FIG. 6, if it is determined at Block 614 that the test subject did not want to seek help in the past, then it is determined at Block 639 whether or not the test subject ever did seek help. If, it is determined at Block 639 that the test subject has not ever sought help, then the test subject may be categorized as non-clinical at Block 638. If, however, it is determined at Block 639 that the test subject did seek help, then it is determined at Block 649 whether or not the test subject has any mental health history. If it is determined at Block 649 that the test subject does have mental health history, then the test subject may be categorized as clinical at Block 628. If, however, is determined at Block 649 that the test subject does not have a mental health history, then no category is assigned at Block 648 (instead, categorization may be based on special scores from the playlist as described below). After completion of any of the categorization Blocks 628, 638, 648, or 658, the method may continue at Blocks 668 and 678 with scoring (more specifically, scoring of the questionnaire at Block 250 and scoring of the playlist at Block 255, both from the flowchart 200 illustrated in FIG. 2).

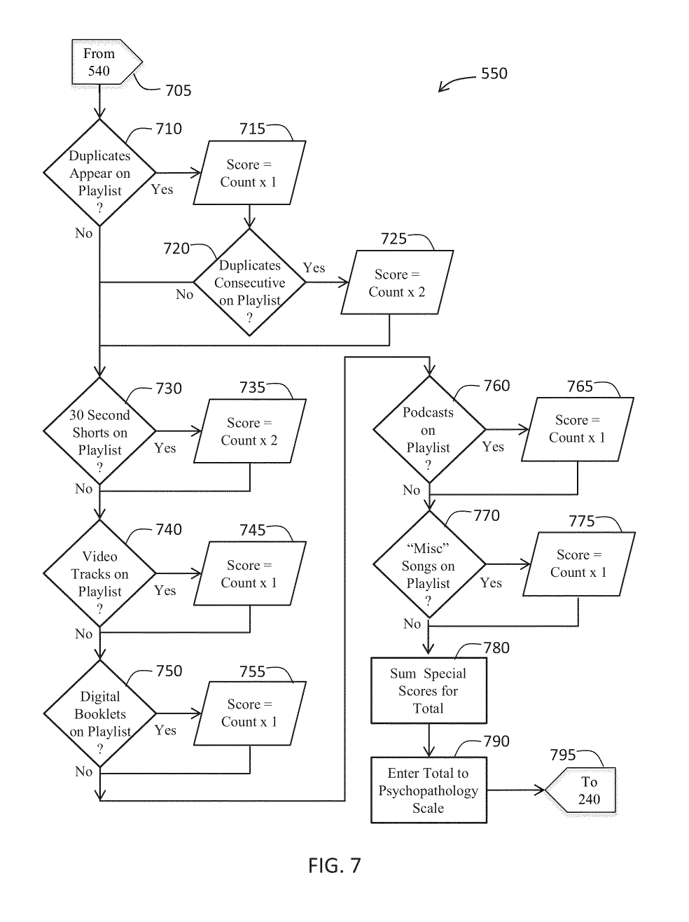

Referring now to the flowchart 550 illustrated in FIG. 7 (which describes further details of Block 550 in the flowchart 230 illustrated in FIG. 5 directed to analyzing special scores), analyzing of special scores as input to the process of categorization of the assessment subject is now described in greater detail. Categorization of the assessment subject may be based on an analysis of playlist characteristics (Block 645 from FIG. 6). More specifically, categorization may be accomplished through analysis of special scores that may characterize a playlist as a whole.

At Block 710, if it is determined that duplicate entries of the same song by the same artist appear on the playlist, then a simple count of the number of duplicated tracks may be generated at Block 715 to flag these events as indicative of the possibility of emotional issues meriting assessment focus. Thereafter, if it is determined at Block 720 that the duplicate entries above appeared consecutively on the playlist, then a higher score, such as duplicated tracks count multiplied by a factor of two (2), may be generated at Block 725 to flag these events as indicative of increased likelihood of mental health issues.

Continuing to refer to FIG. 7, instances of media types of interest on the playlist may be detected, such as tracks of duration that are less than 30 seconds (at Block 730), video tracks (at Block 740), digital booklets (at Block 750), podcasts (at Block 760), and/or are miscellaneous songs that do not fit any other category (at Block 770). A higher score (for example, and without limitation, a track count multiplied by a factor of two (2)), may be generated at Block 730 to flag the presence of 30 second shorts on the playlist as indicative of increased likelihood of mental health issues. A lower score (for example, and without limitation, a track count multiplied by a factor of one (1)), may be generated to flag events that are less indicative of emotional issues, such as the presence of video tracks (at Block 745), digital booklets (at Block 755), podcasts (at Block 765), and miscellaneous songs (at Block 775).

A summation of the scores (Block 780) of the playlist characteristics may be entered into the Psychopathology Scale (Block 790), after which the method may continue at Block 795 with additional description (below) to the step of categorizing an assessment subject (Block 240 from the flowchart 200 of FIG. 2).

### Scoring: Questionnaire

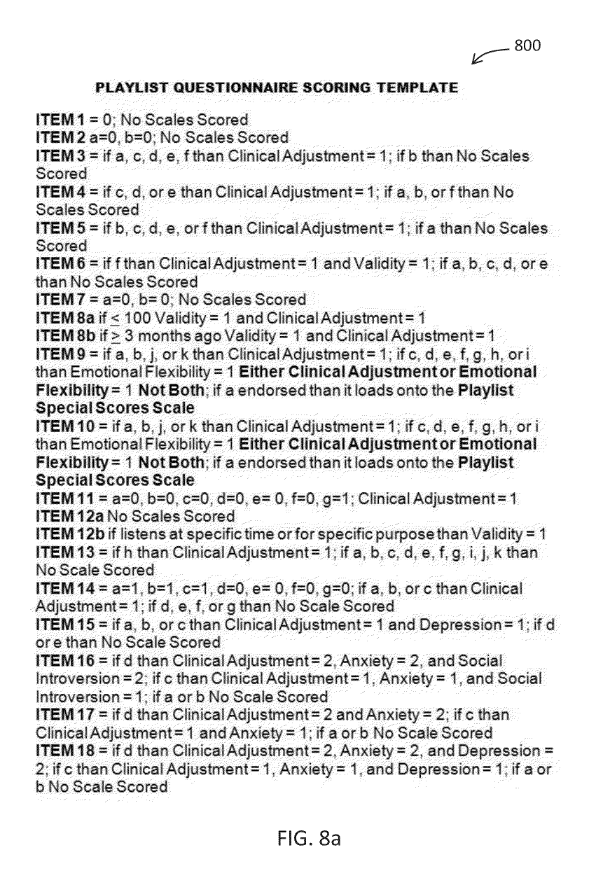

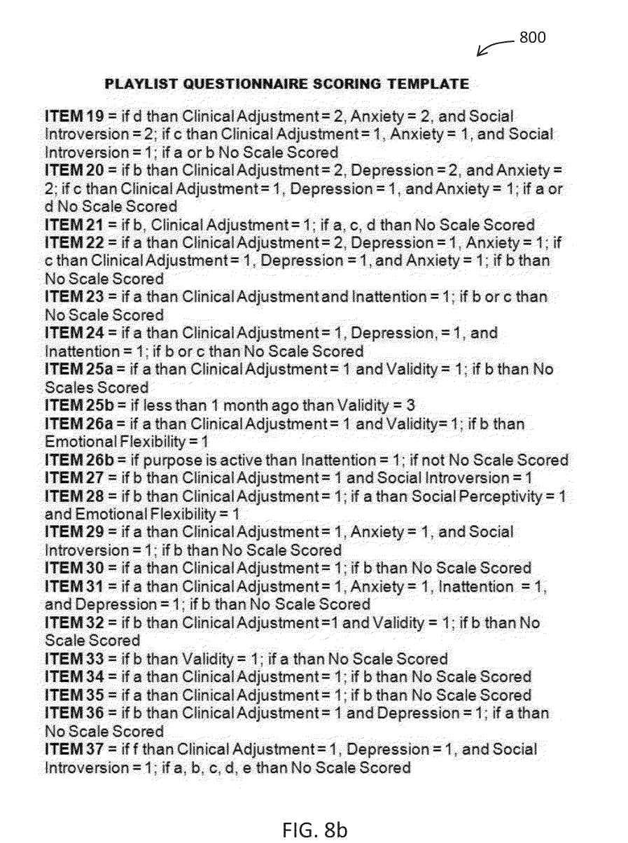

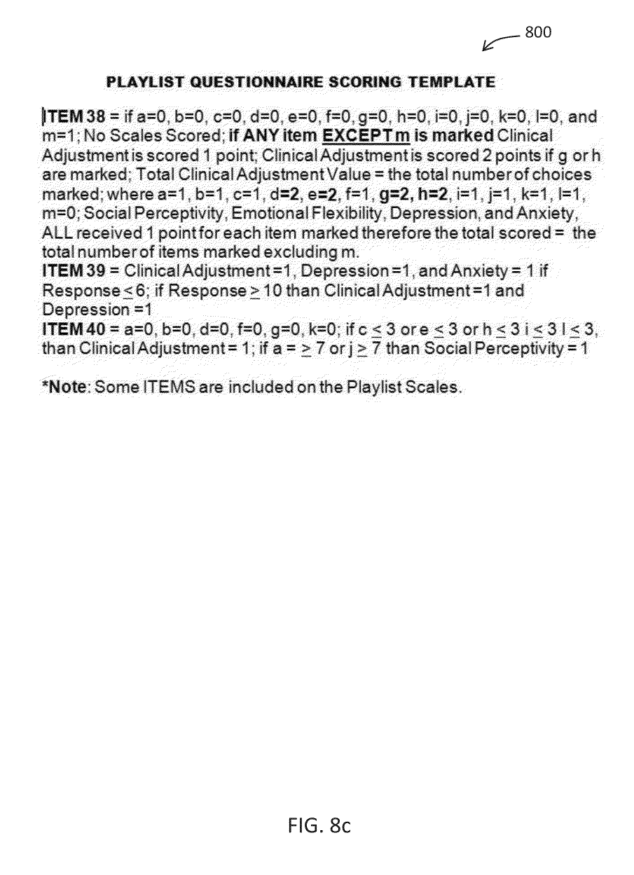

Referring back to FIGS. 1 and 2, performance of personality assessment by the processing module 120 may comprise scoring (Block 250) of the questionnaire responses received in Block 230 based upon qualitative and quantitative control data stored in a database 150. For example, and without limitation, FIGS. 8a through 8c illustrate rules in a Scoring Template 800 that may be used to score responses to the questionnaire illustrated in FIGS. 4a, 4b, and 4c.

The questionnaire 400 may be scored starting with query item tagged ITEM 1 in 800, and continuing for the remaining query items in consecutive order. With the exception of the query item tagged ITEM 2 in 800, each item may load onto one or more Questionnaire Sub-Scales and/or Playlist Sub-Scales. Higher scores on all Sub-Scales (other than the Validity Sub-Scale) may be associated with higher chances of an evaluated individual possessing that characteristic or trait. Sub-Scale Scores may be derived by summing the total points of all the items on that Sub-Scale.

For example, and without limitation, below is a listing of the Questionnaire Sub-Scales (each item may be assumed to scored one point unless otherwise noted below and/or in 800 of FIGS. 8a, 8b, and 8c):

Validity: This Sub-Scale may be a measure of the confidence that the results are an accurate reflection of an individual's response to the questionnaire, and that the submitted playlist is a true representation of the music to which that individual listens. Higher scores may indicate less confidence in the interpretations and hypotheses formed from the results obtained. This Sub-Scale may involve scoring single items, as well as considering responses across multiple items that require consistent responding. Inconsistent response patterns may be examined with regard to query items dispersed throughout the test that may require consistent responses. Failure of an individual to respond consistently where expected may result in higher scores. For example, and without limitation, a response of “Government” to the question, “What is/was your primary extracurricular activity during your most recent school?” may result in a higher score if that same individual responds inconsistently that “Political Environment” rates low in terms of how influential that area of focus is to her life.

Depression: This Sub-scale may measure symptoms of depression according to the latest Diagnostic Manual for Psychiatric Disorders (DSM-IV-TR), as well as symptoms frequently associated with being depressed such as feeling self-conscious and insecure, experiencing social withdrawal, and exhibiting poor work performance. High scores on this scale may represent emotional problems.

Anxiety: This Sub-scale may measure symptoms of anxiety according to the latest Diagnostic Manual for Psychiatric Disorders (DSM-IV-TR), as well as symptoms frequently reported in mental health literature on anxiety disorders such as poor eye contact, difficulty sleeping, and somatic complaints. High scores on this scale may represent emotional problems.

Emotional Flexibility: This Sub-scale may assess an individual's tolerance of and receptivity to variability in emotions, including the ability to regulate emotions and adapt to stress. For example, and without limitation, questions may address various topics including coping skills, role models, family environments, and psychiatric history. High scores may represent health.

Interpersonal Perceptivity: This Sub-scale may assess the degree to which an individual may manifest interpersonal sensitivity and awareness of societal norms, including an individual's interpersonal skills as measured by his experiences, interests, and facility in social situations. For example, and without limitation, questions may relate to factors such as an individual's family experience, willingness to seek out authority figures for assistance and guidance, and interests and comfort in a variety of social contexts ranging from religious environments to the media. High scores may represent health.

Social Introversion: This Sub-scale may measure an individual's level of comfort, interest, and/or fluidity in social interactions. For example, and without limitation, questions such as “Is it hard for you to look into the eyes of other people?” and “Do you feel afraid if you have to speak in front of the class?” may be included on this scale, High scores on this scale may represent emotional problems.

Inattention: This Sub-scale may measure symptoms frequently reported by individuals who have been professionally diagnosed with attention deficit/hyperactivity disorder (ADHD) or who report symptoms consistent with this disorder. Areas that may be assessed include difficulties such as trouble remembering what has been read, academic problems (i.e., poor grades), and disinterest in tasks. High scores on this scale may represent emotional problems.

Self-Intelligence/Clinical Adjustment: This Sub-scale may measure self-knowledge, a tendency to admit vulnerabilities, and the degree to which an individual reports symptomatology. High scores on this scale may represent emotional problems.

Continuing to refer to FIGS. 8a through 8c, a Total Questionnaire Score (TQS) may be calculated by summing Sub-Scale scores from Depression, Anxiety, Inattention & Social Introversion, and then subtracting Sub-Scale scores from Interpersonal Perceptivity and Emotional Flexibility from this sum. Higher scores may be indicative of emotional problems. The TQS also may be used to accurately differentiate individuals who belong to a particular clinical group from those who do not belong to that clinical group. Because the Total Questionnaire Score may be essentially a “weighted” score, the scoring rules may operate to adjust for under and over reporting of problems. Also, TQS may be used as a cutoff or “screening score” in the playlist scoring model to distinguish a non-clinical population from a clinical population.

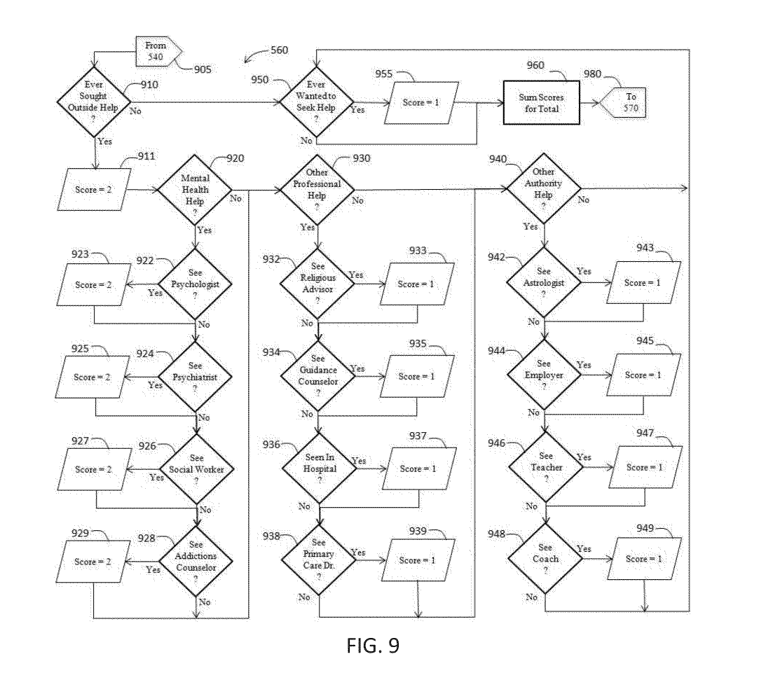

Referring now to the flowchart 560 illustrated in FIG. 9, analysis of critical items (starting at Block 905 which is a continuation from Block 540 of the flowchart 230 of FIG. 5) is now described in greater detail. Critical item analysis may provide information about general functioning of an assessment subject.

At Block 910, if it is determined that the assessment subject ever sought help for emotional issues in the past, then a high score, such as a tally of two (2), may be generated at Block 911 to flag this event as indicative of the presence of mental health issues. Thereafter, instances of seeking help from mental health service providers (Block 920), from other health and counseling professionals (Block 930), and/or from other authorities in the assessment subject's life (Block 940) may be detected. Also, if it is determined at Block 950 that the assessment subject wanted to seek help for emotional issues in the past, but did not, then a lower score, such as a tally of one (1), may be generated at Block 955 to flag this event as indicative of the possibility of mental health issues.

For example, and without limitation, seeing a psychologist (Block 922), a psychiatrist (Block 924), a social worker (Block 926), and/or an addictions counselor (Block 928) may be scored as tallies of two (2) for each of those occurrences (Blocks 923, 925, 927, and 929, respectively). Similarly, seeing a professional such as a religious advisor (Block 932), a guidance counselor (Block 934), a hospital representative (Block 936), and/or a primary care physician (Block 938) may be scored as a tally of one (1) for each of those occurrences (Blocks 933, 935, 937, and 939, respectively). Also, seeing a person in authority such as an astrologist (Block 942), an employer (Block 944), a teacher (Block 946), and/or a coach (Block 948) may be scored as a tally of one (1) for each of those occurrences (Blocks 943, 945, 947, and 949, respectively). If none of the sources of outside help checked at Blocks 920, 930, nor 940 is detected, the method may continue to the Block 950 check without adding to the score based on specific occurrences of sought outside help.

A summation of the scores (Block 960) of the critical items from the questionnaire may be performed, after which the method may continue at Block 980 with additional description (below) to the step of determining whether the playlist supports interpretation (Block 570 from the flowchart 230 of FIG. 5).

The Self-Intelligence Sub-Scale may be loaded based on analysis of the questionnaire either as an alternative to, or in addition to, analysis of the playlist to provide an overall assessment of an individual's self-knowledge and his tendency to be defensive about vulnerabilities. While similar and highly correlated to TQS, comparison of the Self-Intelligence Sub-Scale Score and the TQS may show that TQS will be higher.

### Scoring: Playlist

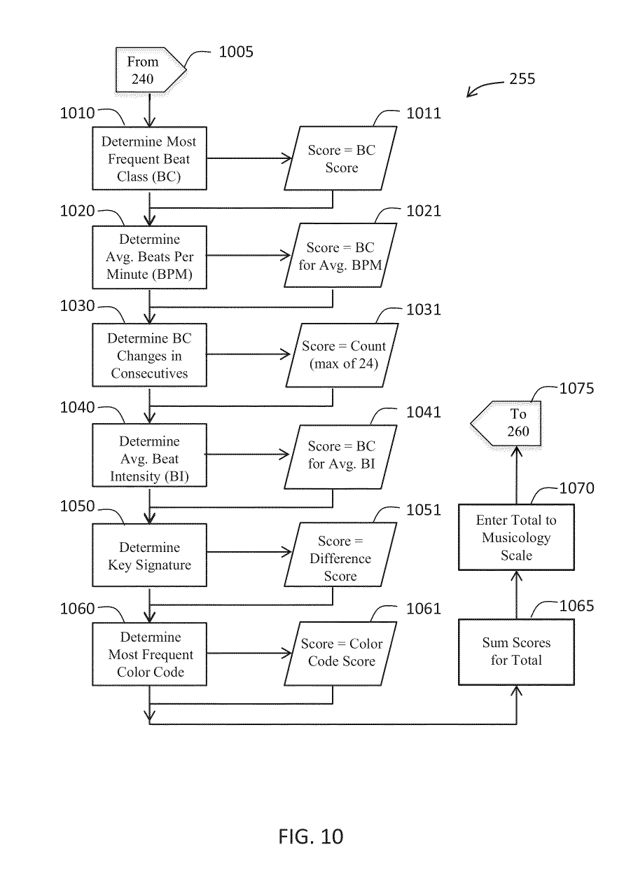

Referring again back to FIGS. 1 and 2, performance of personality assessment by the processing module 120 may comprise scoring (Block 255) of the media selection history received in Block 210 based upon qualitative and quantitative control data stored in a database 150. For example, and without limitation, FIGS. 10 through 16 illustrate rules that may be used to score media playlist entries using a specific method of analysis that may involve analyzing the variables associated with each particular song on the list and also analyzing the variables associated with the overall playlist.

More specifically, analysis of each media playlist entry may load onto one or more Playlist Sub-Scales. Higher scores on all Sub-Scales may be associated with higher chances of an evaluated individual possessing that characteristic or trait. Sub-Scale Scores may be derived by summing the total points of all the items on that Sub-Scale. For example, and without limitation, below is a listing of the Playlist Sub-Scales (each item may be assumed to be scored one point unless otherwise noted).

Each individual song on the playlist may be analyzed for common meta-data that may be categorized as one of verbal and non-verbal content. Subject data for each individual song may come from various sources, for example, and without limitation, a screen shot of a playlist, Internet-based music ontology specifications, and apps available for purchase online or from local retailers.

As illustrated in FIGS. 10 through 13, the non-verbal subject data of interest to assessment of individual songs may be categorized as music variables, artist remarkable features, distinctions, and album information.

Referring now to the flowchart 255 illustrated in FIG. 10, playlist scoring (starting at Block 1005 which is a continuation from Block 240 of the flowchart 200 of FIG. 2) is now described in greater detail. Playlist scoring may comprise an analysis of non-verbal content that may focus on music variables of each song on the playlist.

For example, and without limitation, song beat classifications may be guided by music ontology standards for beats, such as rock, reggae, and hip hop. Beat classifications may be defined as “slow” (20-80 BPM), “moderate” (81-120 BPM), “fast” (121-168 BPM), and “very fast” (greater than 169 BPM). The beat classification found to occur most frequently in the songs on the playlist may be determined at Block 1010, and a beat classification score may be assigned (Block 1011) based on whether the beat classification is slow (score=2), moderate (score=0), fast (score=1), or very fast (score=2).

The beat of each song on the playlist may be analyzed at Block 1020 to determine the average beats per minute (BPM) on the entire playlist. Average BPM may be calculated as the summation of the BPM for all songs on the playlist divided by the total number of songs on the playlist. For example, and without limitation, an average BPM score may be assigned (Block 1021) based on the beat classification defined above that includes the average BPM.

The number of significant beat classification changes (BC) between consecutive songs on the playlist may be determined at Block 1030 from the subject data using the processing module 120. For example, and without limitation, a BC change score may be assigned (Block 1031) as a count of the beat changes for the whole list (after skipping Track 1). To avoid overloading of this variable, the BC change count may be bounded between a minimum of 0 and a maximum of 24.

The beat of each song on the playlist may be analyzed at Block 1040 to determine an average beat intensity (BI) for all songs on the playlist. Song beat intensity (BI) may follow music ontology standards for strength of beat. Average BI may be computed as the summation of the BI for all songs on the playlist divided by the total number of songs on the playlist. For example, and without limitation, an average BI score may be assigned (Block 1041) based on the beat classification defined above that includes the average BI.

Each song may be analyzed at Block 1050 to determine the most frequently occurring signature (e.g., major or minor). A difference score may be computed at Block 1051 as the number of songs on the playlist in a minor key less the number of songs on the playlist in a major key. For example, and without limitation, the difference score for a playlist may be normalized to a score of either 0 or 1.

Also, each song on the playlist may be analyzed to determine most frequently occurring music mood color at Block 1060. For example, and without limitation, color coding of mood traits may employ green to represent “positive energy and very content,” the color red to represent “intense energy,” blue to signify “lethargic, drowsy, and unmotivated,” and the color pink to signify “very depressed.” A color code score may be assigned (Block 1061) based on whether the color code is green (score=0), red (score=1), blue (score=2), or pink (score=3).

A summation of the scores (Block 1065) of the music variables may be entered into the Musicality Scale (Block 1070), after which the method may continue at Block 1075 with additional description (below) to the step of constructing a personality profile (Block 260 from the flowchart 200 of FIG. 2).

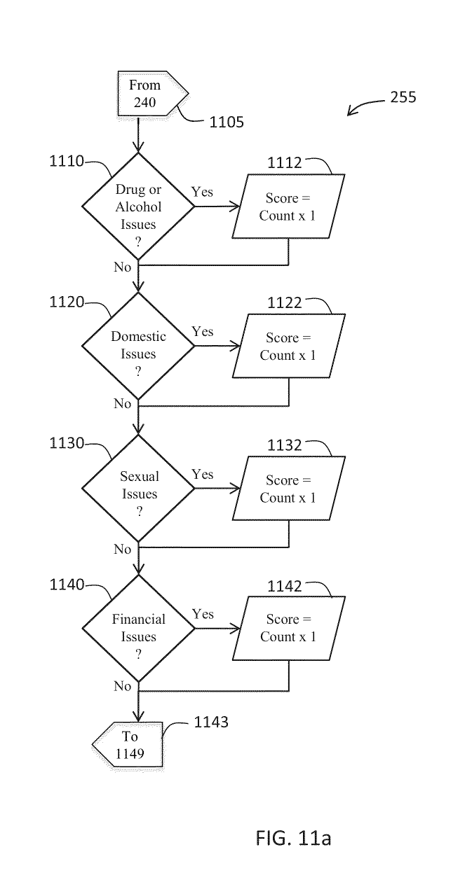

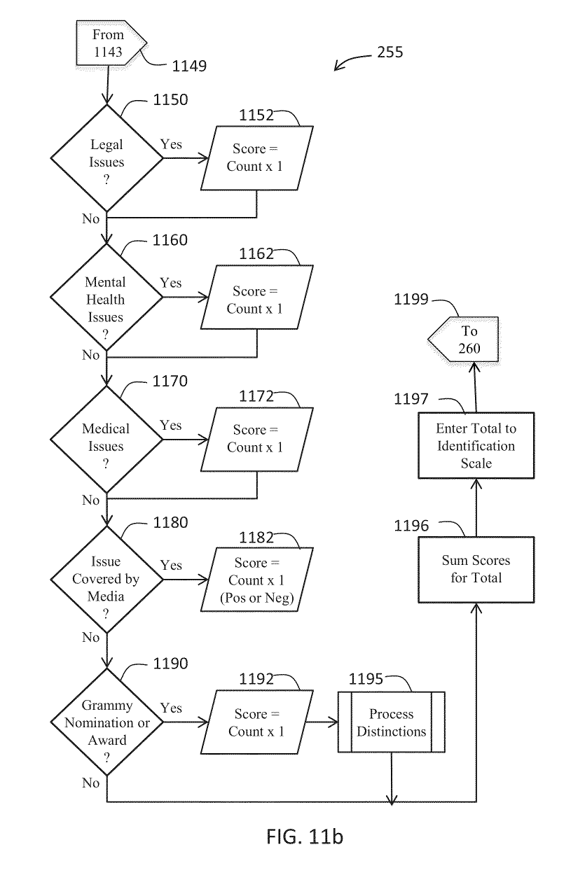

Referring now to the flowchart 255 illustrated in FIGS. 11a and b (description of the playlist scoring from the flowchart 200 of FIG. 2), additional details of scoring are now described. More specifically, the flowchart 255 of FIGS. 11a and 11b describe an analysis of non-verbal content of a song that may focus on remarkable features of the artist. For example, and without limitation, answers to questions about the artist's own background and lifestyle may be analyzed to quantify behavioral influences in focus areas that are significant to developing a personality profile for a follower of the artist. From the start (Block 1105), it is determined whether or not the artist has a history of drug and/or alcohol abuse at Block 1110. If occurrences of such abuse are detected at Block 1110, then a score may be generated at Block 1112 as a single tally for each occurrence. Thereafter, detection of instances of domestic turmoil or violence in the artist's life (Block 1120), sexual indiscretion or deviance on the part of the artist (Block 1130), financial troubles or extravagance on the artist's part (Block 1140), legal problems experienced by the artist (Block 1150), mental health events in the artist's life (Block 1160), and/or medical issues suffered by the artist (Block 1170) may be scored as simple counts of those occurrences (Blocks 1122, 1132, 1142, 1152, 1162, and 1172, respectively).

Continuing at Block 1180, instances of media exposure directed at the artist (either positive or negative) may be counted and scored (Block 1182), for example, and without limitation, as an addition of one (1) for each negative issue covered by the media, and a deduction of one (1) for each positive issue covered by the media.

At Block 1190, detection of instances of recognition bestowed on the artist, referred to herein as distinctions, may occur. Distinctions may be counted and scored at Block 1192, for example, and without limitation, for each nomination for and/or winning of a Grammy Award. Detection and scoring of such an award may also spawn more detailed processing of distinctions (Block 1195), which is described in greater detail below.

A summation of the scores 1196 (e.g., number of occurrences) of the artist remarkable features may be entered into the Identification Scale 1197, after which the method continues at Block 1199 with additional description (below) to the step of constructing a personality profile (Block 260 from the flowchart 200 of FIG. 2).

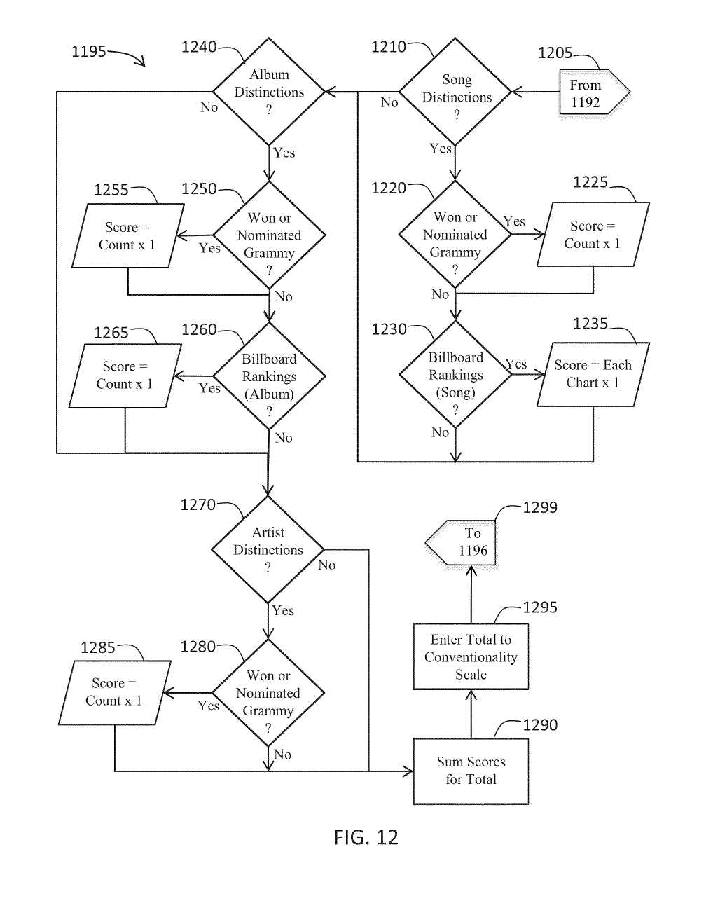

Indications of the critical and/or commercial success of a song on a playlist may be analyzed to quantify a disposition of a consumer of the song to conform to convention. Referring now to the flowchart 1195 illustrated in FIG. 12 (description of the processing of distinctions from the flowchart 255 of FIG. 11b), analysis of non-verbal content of a song may focus on processing such distinctions related to a song. Starting at Block 1205 (which is a continuation from the score being assigned at Block 1192 of the flowchart 255 illustrated in FIG. 11b), it is determined at Block 1210 whether or not the song exhibits any distinctions. Similarly, it may be determined at Block 1240 whether or not the album that includes the song exhibits any distinctions, and at Block 1270 if the artist who recorded the song exhibits any distinctions. If not the song, nor the album, nor the artist exhibits any distinctions related to the playlist entry, then analysis of this focus area may end at Block 1299 after no scores are detected at Block 1290 nor added to the Conventionality Sub-Scale ab Block 1295. However, if the song, album, and/or artist have been nominated for or won any Grammy Awards (Blocks 1220, 1250, and 1280, respectively), then each award may be counted and scored, for example, and without limitation, as an addition of one (1) for each such event recognizing one or more of the song (at Block 1225), the album (at Block 1255), and the artist (at Block 1285).

Continuing to refer to FIG. 12, at Block 1230 it is determined whether or not the song has the distinction of appearance on the Billboard rankings. Similarly, at Block 1260, it is determined whether or not the album has appeared on the Billboard rankings. Each such appearance may be counted and scored, for example, and without limitation, as an addition of one (1) for each such event involving one or more of the song (at Block 1235) and the album (at Block 1265). For example, and without limitation, a distinction score may load for a ranking for each playlist entry on one or more popular charts such as the U.S. Billboard Pop 100, the U.S. Billboard Hot 100, or other song distinction lists. A distinction score may also load for a ranking of a song on five (5) or more charts. Also, a distinction score may load for a ranking of the album from which a playlist entry originates on the U.S. Billboard 200 or other album distinction list.

At Block 1290, a summation of the scores (e.g., number of award occurrences and chart rankings) for distinctions may be computed for subsequent entry into the Conventionality Scale (Block 1295), after which the subprocess may return at Block 1299 to complete summation of total scores for artist remarkable features (Block 1196 from FIG. 11b).

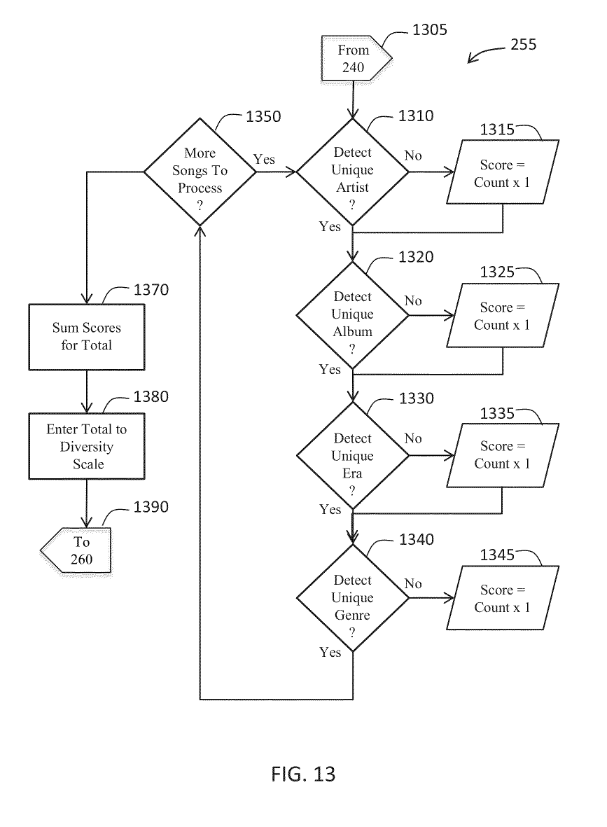

Referring now to a continuation of the flowchart 255 illustrated in FIG. 13, analysis of non-verbal content of a song may focus on information about the album on which the song was released. For example, and without limitation, information for the album-specific variables may be found on a screenshot of the playlist. Starting at Block 1305, detection of album information may include detection of each appearance in a playlist of an individual artist (Block 1310), of an individual album identifier (Block 1320), of a particular the release date/era (Block 1330), and of a particular music genre (Block 1340). Each such appearance may be counted and scored, for example, and without limitation, as an addition of one (1) for each detection of one or more of an artist (at Block 1315), an album (at Block 1325), an era (at Block 1335), and a genre (at Block 1345). The preceding detection and scoring procedures may be applied until every song present in a playlist has been processed, as detected at Block 1350. To prevent inflation of scores, Block 1350 may employ rule out questions to prevent double counting of artists/bands, albums, eras, and/or genres that may have been scored during album information analysis of a prior song.

A summation of the scores 1370 (e.g., number of occurrences) for the album information may be entered into the playlist Diversity Scale (Block 1380), after which the subprocess may return at Block 1390 (to Block 260 from FIG. 2 to construct a personality profile).

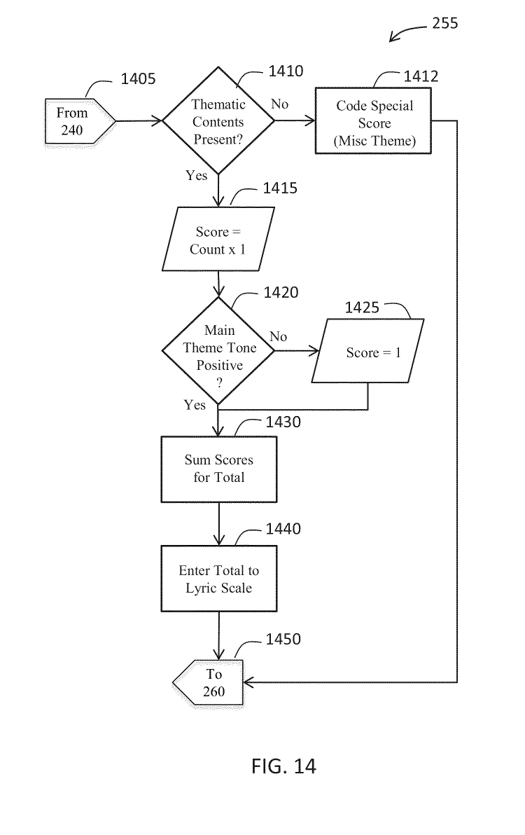

As illustrated in FIGS. 14 through 16, the verbal subject data of interest to assessment of individual songs may be analyzed based on three (3) levels of lyric abstraction, moving from general to specific: lyrical theme, repeated phrases, and word count.

Referring now to FIG. 14, playlist scoring 255 may comprise analysis of verbal content may focus on the lyrical theme(s) present in each song on the playlist. For example, and without limitation, the lyrics of a particular song may contain one or more of the following types of thematic contents: inspirational/hope/empowerment, suicide, homicide, violence, peace/love, sex/relationship, political/philosophical, alienated/isolated, inner conflict/angst, and heartbreak/loss/regret. After the subprocess begins at Block 1405, detection of thematic contents at Block 1410 may include detection of each appearance in a playlist of a song of a known thematic content type. Each such appearance may be counted and scored, for example, and without limitation, as an addition of one (1) for each detection of a particular thematic content type (at Block 1415).

At Block 1420, the affective tone of the main lyrical theme of each song may be determined to be either positive or negative 1420, and may be counted and scored, for example, and without limitation, as an addition of one (1) for each detection of a negative tone (at Block 1425). A total lyrical theme score for all songs 1430 may be summed at Block 1430 before being entered into the Lyric Scale at Block 1440. The subprocess may then terminate at Block 1450.

In an alternative embodiment, a special score may be coded at Block 1412 if none of the types of thematic content supported by the profiling system 100 are detected at Block 1410. In such a scenario, the subprocess may terminate at Block 1450, leaving lyrical analysis to be replaced by summing of critical items from the questionnaire.

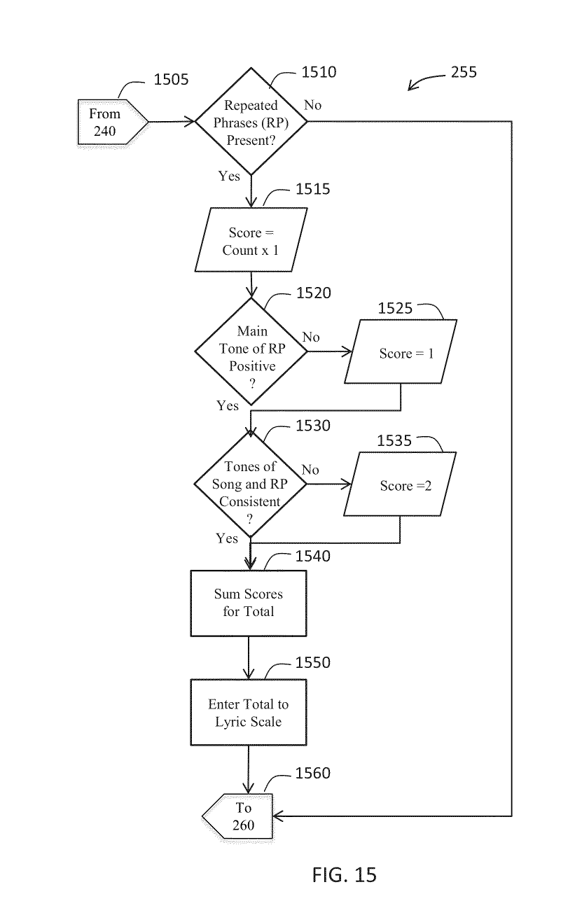

Referring now to FIG. 15, playlist scoring 255 may comprise analysis of verbal content present in each song on the playlist. For example, and without limitation, the analysis of verbal content may focus on the presence and character of repeated phrases in the lyrics of a song. After the subprocess begins at Block 1505, detection of repeated phrases at Block 1510 may result in multiple and different repeated phrases being counted and scored, for example, and without limitation, as an addition of one (1) for each detection of a particular repeated phrase (at Block 1515). Alternatively, if no repeated phrases are detected at Block 1510, the subprocess may then terminate at Block 1560.

At Block 1520, the affective tone of a repeated phrase in each song may be determined to be either positive or negative 1520, and may be counted and scored, for example, and without limitation, as an addition of one (1) for each detection of a negative tone (at Block 1525). Also, if the affective tone of a repeated phrase detected in Block 1520 is found to be inconsistent with the affective tone of the main lyrical theme of the song detected in Block 1420 of FIG. 14, this inconsistency may be scored, for example, and without limitation, as an addition of two (2) for each detection of an inconsistent tone (at Block 1535). A total repeated phrase score for all songs may be summed at Block 1540 before being entered into the Lyric Scale at Block 1550. The subprocess may then terminate at Block 1560.

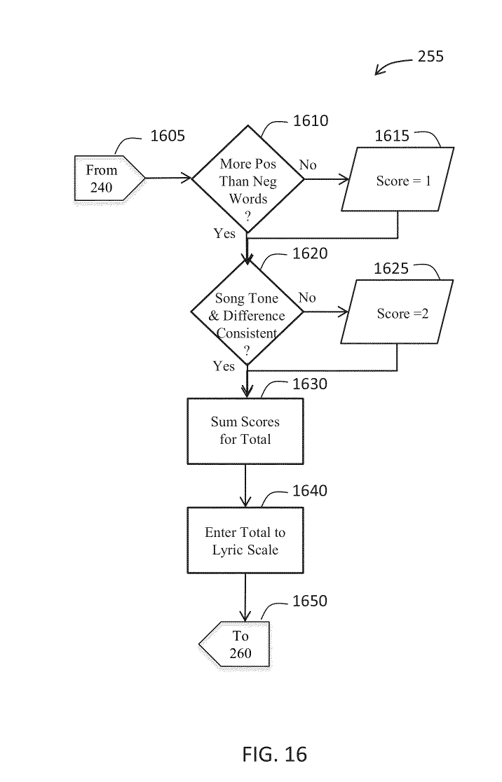

Referring now to FIG. 16, playlist scoring 255 may comprise analysis of verbal content present in each song on the playlist. For example, and without limitation, the analysis of verbal content may focus on a count of words in the lyrics of a song. After the subprocess begins at Block 1605, computing the ratio of positive affective words to negative affective words detected in the song at Block 1610 may result in net negative word counts being scored, for example, and without limitation, as an addition of one (1) for each detection of negative lyrics (at Block 1615). Also, if the affective difference score computed in Block 1615 is found at Block 1620 to be inconsistent with the affective tone of the main lyrical theme of the song detected in Block 1420 of FIG. 14, this inconsistency may be scored, for example, and without limitation, as an addition of two (2) for each detection of such inconsistency (at Block 1625). A total word count score for all songs may be summed at Block 1630 before being entered into the Lyric Scale at Block 1640. The subprocess may then terminate at Block 1650.

### Personality Profile

Referring again to FIGS. 1 and 2, construction of a personality profile (Block 260) by the output module 130 may comprise interpreting the scores computed during analysis of the questionnaire responses and the media selection history. Such dual-media analysis may operate to mitigate the risk to validity of results posed by self-report measures like the Playlist Questionnaire when employed without a quality check.

By analyzing the media selection history for revealed preference factors such as the most commonly played songs and combining those results with the questionnaire, a detailed and revealing personality profile may emerge that may temper or even overcome self-report bias. The present invention 100 assigns questionnaire variables, individual song variables, and to specific Sub-Scales (i.e., Questionnaire Sub-Scales or Playlist Sub-Scales). The playlist may be scored with over thirty variables. Scores on a playlist may be obtained by scoring each individual song after an Individual has been categorized based upon responses to critical items in the Questionnaire or by a scoring of the individual's playlist for Special Scores. Once categorized, then each song may be scored in a systematic way. The following is a list of the Playlist Sub-Scales, their definitions, and items:

Psychopathology: This Playlist Sub-Scale may measure psychopathology/deviance, for the purpose of classifying individuals as being most similar to a clinical sample using a playlist. High scores may indicate that a person matches the scores obtained by a clinical sample. High scores on this scale may also represent emotional problems. The score for the scale may be obtained by viewing the playlist screenshot as well as by analyzing all songs for lyrical thematic content. For example, and without limitation, the Psychopathology Playlist Sub-Scale score may be computed as a function of Special Scores Composite Score, Validity Score (Questionnaire), Negative Mean Difference Between Era and Date of Birth Score, Homicide Index Positive if at least 7 songs, Violence Index Positive if at least 10 songs, Suicide Index if at least 3 songs, Clinical Adjustment Score (Questionnaire) at least 35, detection of at least 3 Songs that meet criteria for Discontinuation Rule, and the Number of Perseverations on the Playlist.

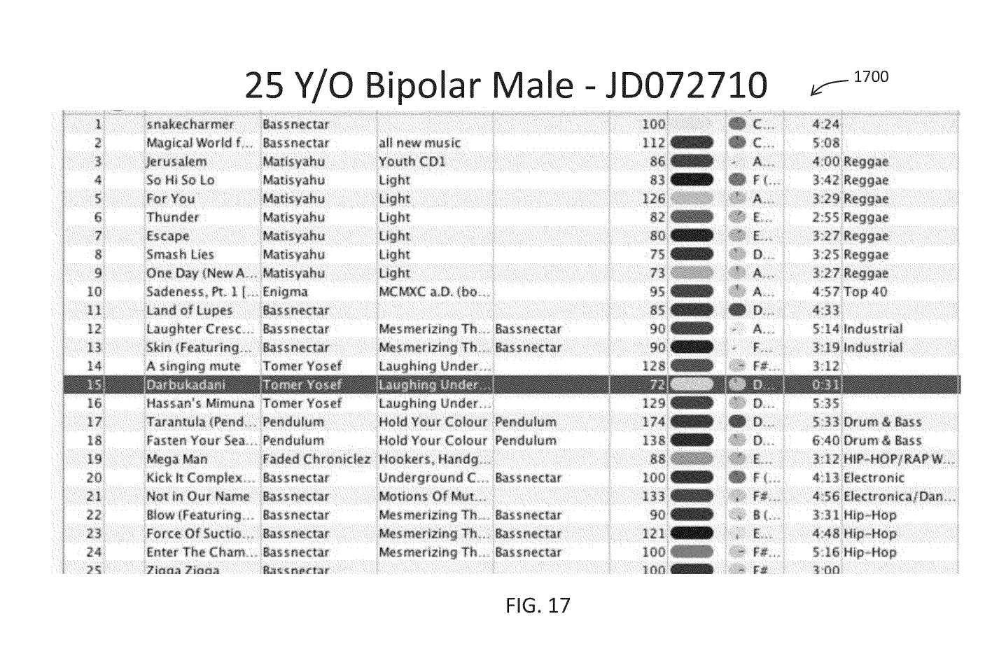

Identification: This Playlist Sub-Scale may measure conscious and unconscious emotional problems and an individual's potential capacity to mediate the effects of these issues. High scores on this scale may represent emotional problems. The score for this Sub-Scale may be obtained by summing two types of Remarkable Features (RF) scores. The first RF Type is a means of determining whether or not the Remarkable Features scored were significant because they were either positive (i.e., healthy) or negative (i.e., unhealthy). Therefore, this variable is particularly important because it determines whether an individual is drawn to artist/bands for negative (or less healthy) reasons. The second RF Type provides the number of Remarkable Features. An aspect to this scale is examination of each of the RFs listed to determine if the RF most scored on an individual's playlist provides information about what the individual may be experiencing. For example, and without limitation, the RF that receives the highest score is likely to be an issue with which the individual has problems either directly or indirectly. The Identification Playlist Sub-Scale score may be computed as a function of a Remarkable Features Composite Score, a Correlation Between Musicality Scale Score and Clinical Adjustment Score, a Correlation Between Lyric Scale Score and Clinical Adjustment Score. Higher scores may be indicative of stronger associations with that trait or characteristic. For example, and without limitation, FIG. 17 illustrates a playlist characterized by a Identification Scale score as follows:

A. ARTIST ETOH=TOTAL SCORE FOR LIST=1

B. ARTIST FINANCIAL=SCORE FOR LIST=0

C. ARTIST SEXUAL=TOTAL SCORE FOR LIST=0

D. ARTIST MISCELLANEOUS+/−=LIST AND SPECIFY=3 (2 Religion, 1 Activist)

E. ARTIST=GRAMMY SCORE FOR LIST=1

F. ARTIST DOMESTIC=TOTAL SCORE FOR LIST=1

G. ARTIST LEGAL=TOTAL SCORE FOR LIST=2

H. ARTIST MH=TOTAL SCORE FOR LIST=1,

I. ARTIST MEDICAL=TOTAL SCORE=1

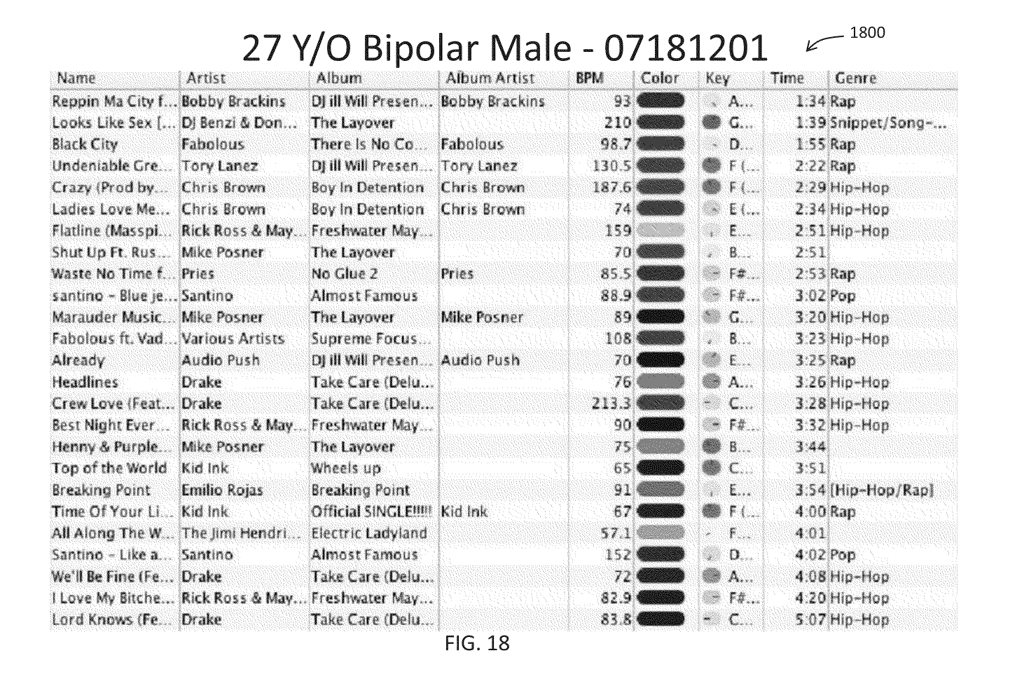

Musicality: This Playlist Sub-Scale may measure the underlying mood state created by song based upon non-verbal communication. For example, and without limitation, the Musicality Playlist Sub-Scale score may be computed as a function of the Difference score obtained by subtracting all songs in Minor Key Signature from All Songs in Major Key Signature, the Most Frequently Scored Color Code for all songs, the most frequently scored BPM based upon taking the average BPM for all songs and determining the classification of that beat, and the Most Frequently scored Beat Classification. For example, and without limitation, FIG. 18 illustrates a playlist characterized by a Musicality scale score computed as follows:

A. BPM=MEAN BPM FOR PLAYLIST (sum of all BPM's for Scored Tracks divided by No. Tracks on Playlist)=103.6

B. BEAT CLASSIFICATION=MOST FREQUENT beat classification scored ON LIST=MOD

C. BEAT CHANGE=NO. OF TIMES ON LIST SIG. DIFF. IN BPM B/W CONSECUTIVE SONGS=14

D. BI=MEAN BI FOR PLAYLIST (sum of BI's for Scored Tracks divided by No. Tracks on Playlist)=49

E. KEY SIGNATURE=NO. MINOR KEY: NO. MAJOR KEY=15:10

F. COLOR CODE=MOST FREQUENT COLOR ASSIGNED TO SONGS ON LIST=DARK BLUE/PURPLE

Conventionality: This Playlist Sub-Scale may measure reality testing (i.e., does the individual see things as others do) and peer group identification. High Scores may represent healthy functioning. For example, and without limitation, a Conventionality Playlist Sub-Scale score may be computed as a function of a Distinction Composite Score and of Influence Ratings from the questionnaire. Billboard Rankings may also load the Distinctions Sub-Scale Score. A higher Distinctions Score may mean that the individual is picking more songs that are typical and “popular” amongst her peer group. This scale is also one that may be viewed in conjunction with other Sub-Scales that are associated with emotional problems. It may be a sign of problems if an individual obtained high scores on sub-scales that are associated with emotional problems and a low score on this scale, because this would suggest that not only does an individual seem to have problems, but that individual may also have poor reality testing. In such a case, further understanding of the individual's playlist and questionnaire scores may be important to help rule out additional signs of possible psychosis.

Diversity: This Playlist Sub-Scale may measure psychological flexibility, life experience and exposure, and openness. High Scores may represent healthy functioning. For example, and without limitation, the Diversity Playlist Sub-scale score may be computed as a function of Composite Score Artist, Composite Score Album, Composite Score Genre, and Composite Score Era.

Lyric Scale: This Playlist Sub-Scale may measure underlying mood state and the coexistence of positive and negative feelings simultaneously creating tension and pull in opposite directions. For example, and without limitation, the Lyric Playlist Sub-Scale score may be computed as a function of Critical Composite Score Positive, Critical Composite Score Negative, Mean Proportion of Positive Critical Items to Word Count, Mean Proportion of Negative Critical Items to Word Count, Mean Valence Score (Ratio of Positive to Negative Critical Items), Mean Intensity of Affective Tone of Lyric, Repeated Phrases Composite Score Positive, and Repeated Phrases Composite Score Negative.

Aggression Scale—This Playlist Sub-Scale may measure an individual's preference for content that has features associated with hostility and aggression. High scores on this scale may represent emotional problems. For example, and without limitation, the Aggression Playlist Sub-scale score may be computed as a function of Homicide Index Positive if at least 7 songs, Violence Index Positive if at least 10 songs, Beat Classification at least 121, and Mode Color Code.

Depression Scale—This Playlist Scale may measure self-esteem, depression, and impulse control. High scores on this scale represent emotional problems. For example, and without limitation, the Depression Playlist Sub-scale score may be computed as a function of Suicide Index if at least 3, Composite Score Thematic Content, Depression Score (Questionnaire), Beat Classification less than 80, Ratio of Minor to Major Key Signature, Mode Color Code, and a count of songs with a main lyrical theme of either Heartbreak/Loss/Regret, Inner Conflict/Angst, Alienation/isolation, or Sex/Relationships.

General Wellness Scale—This Playlist Scale may measure resiliency, internal stability, and positive outlook. High Scores may represent healthy functioning. For example, and without limitation, the General Wellness Playlist Sub-scale score may be computed as a function of Composite Score Thematic Content Hope/Love, Clinical Adjustment Score (Questionnaire) less than 20, Affective Flexibility Score (Questionnaire), Positive Mean Valence Score, Ratio of Minor to Major Key Signature, and Mode Color Code (Perseverations, Podcasts, Audiobooks).

Social Consciousness Scale—This Playlist Scale may measure social awareness and empathy, which may be computed as a function of Interpersonal Perceptivity Score (Questionnaire), Composite Score, Thematic Content, and Political Awareness.

ADHD Scale—This Playlist Sub-Scale may measure attention, impulsivity, sensation seeking, and threshold for stimulation. High scores on this scale represent emotional problems. For example, and without limitation, the ADHD Scale may be computed as a function of Inattentiveness Score (Questionnaire), Mean BPM, Mean Beat Intensity, Total Number of Beat Changes Between Consecutive Songs, Beat Classification less than 121, Composite Score Artist, and Mode Color Code.

Referring again to FIGS. 1 and 2, construction of an individual's customized personality profile (Block 260) by the output module 130 may conclude by adding a Total Playlist Score (TPS) and the individual Playlist Scale Scores to create an aggregate scale score. An overall aggregate score may be generated by taking the summation of the Overall Playlist Score and the Total Questionnaire Score. The TPS may determine whether an individual is more similar to a non-clinical sample or a clinical sample. More specifically, the TPS may indicate whether an individual's questionnaire responses and music preferences match the responses that have been given by different samples taken from non-clinical groups of individuals between the ages of fifteen and thirty or various types of clinical samples between the ages of fifteen and thirty years. Higher TPS scores may have been obtained by individuals with more severe psychiatric diagnoses.

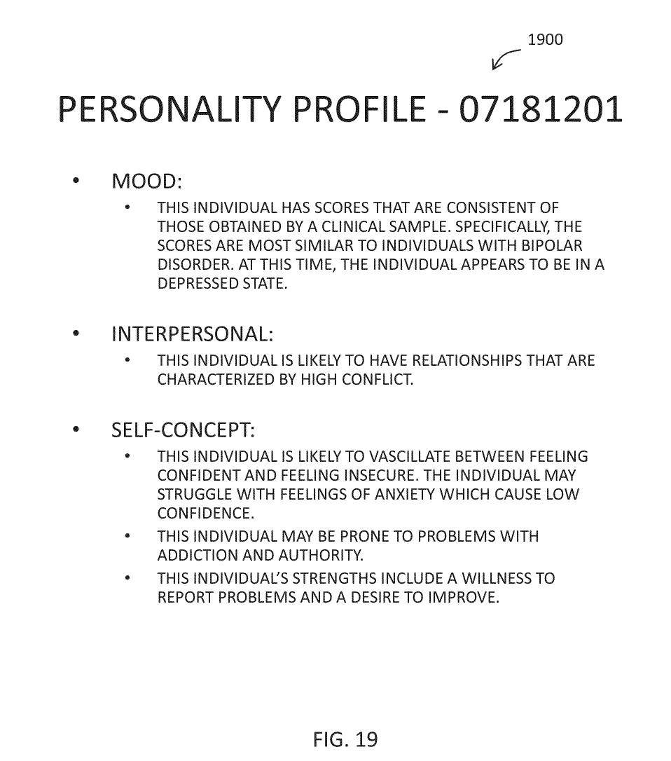

For example, and without limitation, FIG. 19 illustrates a sample personality profile that may be generated by the personality profiling system 100.

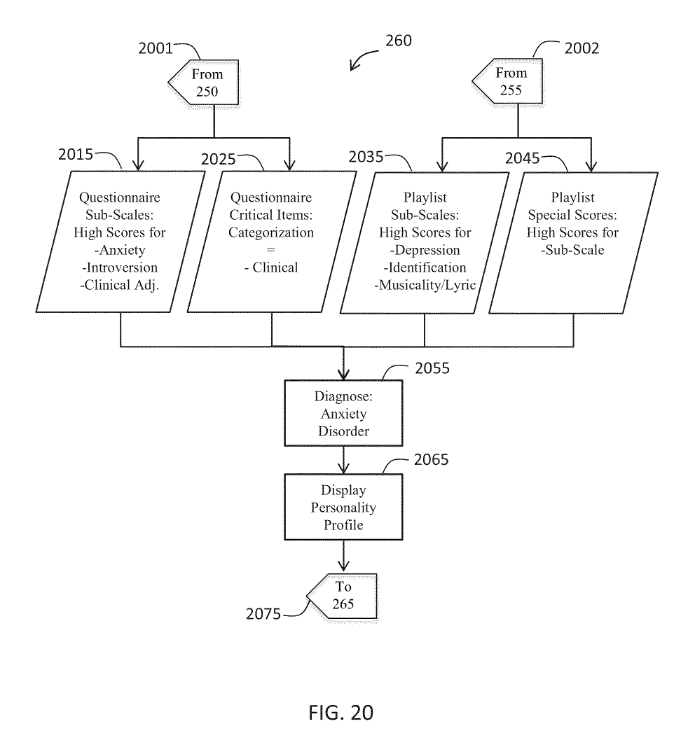

Referring now to the flowchart 260 illustrated in FIG. 20, the step of display of a diagnosis is described in greater detail. More specifically, the flowchart 260 illustrated in FIG. 20 merely provides enhanced detail of the step of constructing a personality profile (Block 260 in the flowchart 200 of FIG. 2) and, as such, is similarly labeled. From the start at which questionnaire analysis results (Block 2001) and playlist analysis results (Block 2002) are provided to the output module (130 of FIG. 1), the input Questionnaire Sub-Scales (Block 2015), input Questionnaire Critical Items (Block 2025), input Playlist Sub-Scales (Block 2035), and input Playlist Special Scores (Block 2045) may be correlated to control data for purposes of matching those data to a mental health diagnosis at Block 2055. In the example of FIG. 20, Block 2015 may present high scores for the Anxiety, Introversion, and Clinical Adjustment sub-scales of the questionnaire. Also, Block 2025 may present a categorization of the assessment subject as being clinical. Block 2035 may present high scores for the Depression, Identification, and Lyric variable of the Musicality sub-scales of the playlist. Also, Block 2045 may present the special scores of the playlist as being high. The output module may generate a diagnosis at Block 2055 of “Anxiety Disorder,” and may display that diagnosis and supporting background information as part of a personality profile at Block 2065, before the method terminates (Block 2075).

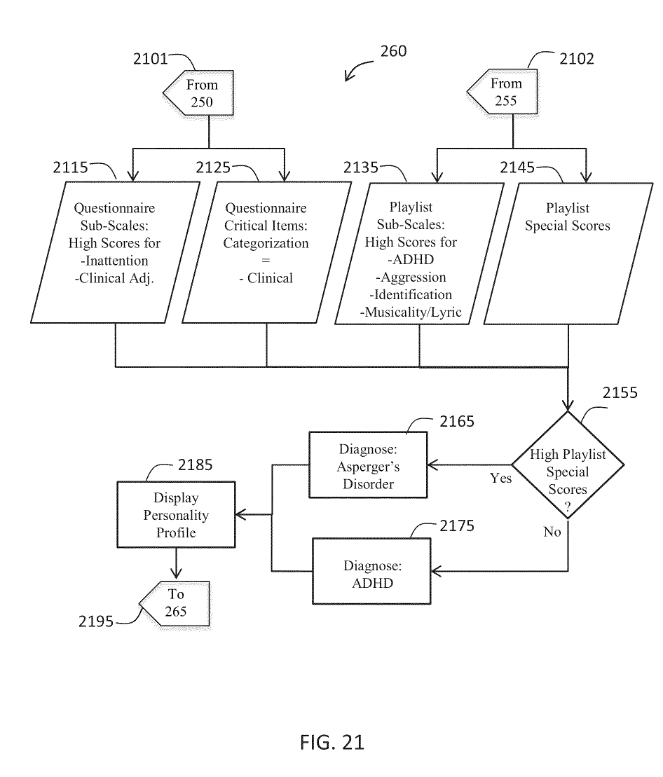

Referring now to the flowchart 260 illustrated in FIG. 21, another example of the step of displaying a diagnosis is described in greater detail. The flowchart 260 illustrated in FIG. 21 provides enhanced detail of the step of constructing a personality profile (Block 260 in the flowchart 200 of FIG. 2) and is similarly labeled. From the start at which questionnaire analysis results (Block 2101) and playlist analysis results (Block 2102) are provided to the output module (130 of FIG. 1), the input Questionnaire Sub-Scales (Block 2115), input Questionnaire Critical Items (Block 2125), input Playlist Sub-Scales (Block 2135), and input Playlist Special Scores (Block 2145) may be correlated to control data for purposes of matching those data to a mental health diagnosis at Block 2155. In the example of FIG. 21, Block 2115 may present high scores for the Inattention and Clinical Adjustment sub-scales of the questionnaire. Also, Block 2125 may present a categorization of the assessment subject as being clinical. Block 2135 may present high scores for the ADHD, Aggression, Identification, and Lyric variable of the Musicality sub-scales of the playlist. At Block 2155, it may be determined whether the special scores of the playlist are presented as being high. If the special scores are high, then the output module may generate a diagnosis at Block 2165 of “Asperger's Disorder,” and may display that diagnosis and supporting background information as part of a personality profile at Block 2185, before the method terminates (Block 2195). If, however, the special scores are not high, then the output module may generate a diagnosis at Block 2175 of “ADHD,” and may display that diagnosis and supporting background information as part of a personality profile at Block 2185, before the method terminates (Block 2195).

While the preceding description shows and describes one or more embodiments, it will be understood by those skilled in the art that various changes in form and detail may be made therein without departing from the spirit and scope of the present disclosure. For example, additional mental health diagnoses may be supported. In addition, various steps of the described methods may be executed in a different order or executed sequentially, combined, further divided, replaced with alternate steps, or removed entirely. Also, various functions illustrated in the methods or described elsewhere in the disclosure may be combined to provide additional and/or alternate functions.

As described, some or all of the steps of each method may be implemented in the form of computer executable software instructions. Furthermore, the instructions may be located on a server that is accessible to many different clients, may be located on a single computer that is available to a user, or may be located at different locations. Therefore, the claims should be interpreted in a broad manner, consistent with the present disclosure. While various embodiments have been described for purposes of this disclosure, numerous changes and modifications will be apparent to those of ordinary skill in the art. Such changes and modifications are encompassed within the spirit of this invention as defined by the claims.

### Computer Implementation

Embodiments of the present invention are described herein in the context of a system of computers, servers, and software. Those of ordinary skill in the art will realize that the following embodiments of the present invention are only illustrative and are not intended to be limiting in any way. Other embodiments of the present invention will readily suggest themselves to such skilled persons having the benefit of this disclosure.

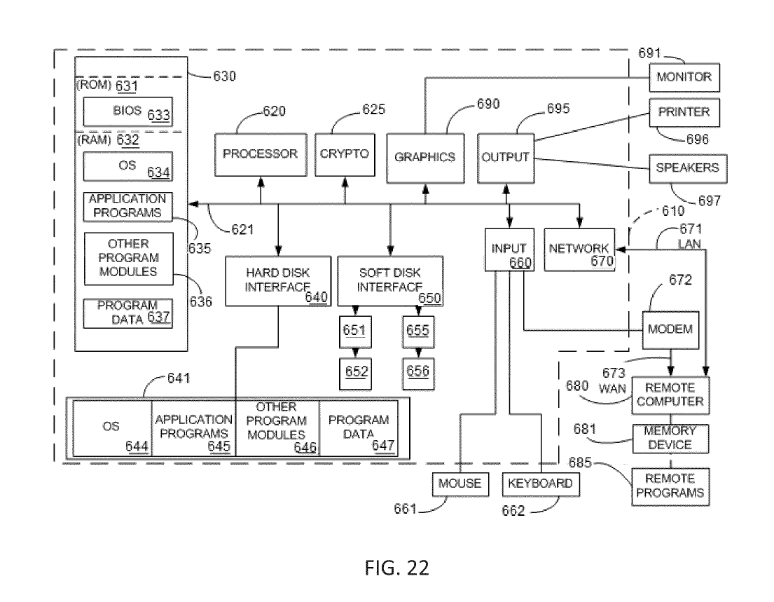

A skilled artisan will note that one or more of the aspects of the present invention may be performed on a computing device, including mobile devices. The skilled artisan will also note that a computing device may be understood to be any device having a processor, memory unit, input, and output. This may include, but is not intended to be limited to, cellular phones, smart phones, tablet personal computers (PCs), laptop computers, desktop computers, personal digital assistants (PDAs), etc. FIG. 22 illustrates a model computing device in the form of a computer 610, which is capable of performing one or more computer-implemented steps in practicing the method aspects of the present invention. Components of the computer 610 may include, but are not limited to, a processing unit 620, a system memory 630, and a system bus 621 that couples various system components including the system memory to the processing unit 620. The system bus 621 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI).

The computer 610 may also include a cryptographic unit 625. Briefly, the cryptographic unit 625 has a calculation function that may be used to verify digital signatures, calculate hashes, digitally sign hash values, and encrypt or decrypt data. The cryptographic unit 625 may also have a protected memory for storing keys and other secret data. In other embodiments, the functions of the cryptographic unit may be instantiated in software and run via the operating system.

A computer 610 typically includes a variety of computer readable media. Computer readable media can be any available media that can be accessed by a computer 610 and includes both volatile and nonvolatile media, removable and non-removable media. By way of example, and not limitation, computer readable media may include computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, FLASH memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by a computer 610. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared and other wireless media. Combinations of any of the above should also be included within the scope of computer readable media.

The system memory 630 includes computer storage media in the form of volatile and/or nonvolatile memory such as read only memory (ROM) 631 and random access memory (RAM) 632. A basic input/output system 633 (BIOS), containing the basic routines that help to transfer information between elements within computer 610, such as during start-up, is typically stored in ROM 631. RAM 632 typically contains data and/or program modules that are immediately accessible to and/or presently being operated on by processing unit 620. By way of example, and not limitation, FIG. 22 illustrates an operating system (OS) 634, application programs 635, other program modules 636, and program data 637.

The computer 610 may also include other removable/non-removable, volatile/nonvolatile computer storage media. By way of example only, FIG. 22 illustrates a hard disk drive 641 that reads from or writes to non-removable, nonvolatile magnetic media, a magnetic disk drive 651 that reads from or writes to a removable, nonvolatile magnetic disk 652, and an optical disk drive 655 that reads from or writes to a removable, nonvolatile optical disk 656 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 641 is typically connected to the system bus 621 through a non-removable memory interface such as interface 640, and magnetic disk drive 651 and optical disk drive 655 are typically connected to the system bus 621 by a removable memory interface, such as interface 650.

The drives, and their associated computer storage media discussed above and illustrated in FIG. 22, provide storage of computer readable instructions, data structures, program modules and other data for the computer 610. In FIG. 22, for example, hard disk drive 641 is illustrated as storing an OS 644, application programs 645, other program modules 646, and program data 647. Note that these components can either be the same as or different from OS 634, application programs 635, other program modules 636, and program data 637. The OS 644, application programs 645, other program modules 646, and program data 647 are given different numbers here to illustrate that, at a minimum, they may be different copies. A user may enter commands and information into the computer 610 through input devices such as a keyboard 662 and cursor control device 661, commonly referred to as a mouse, trackball or touch pad. Other input devices (not shown) may include a microphone, joystick, game pad, satellite dish, scanner, or the like. These and other input devices are often connected to the processing unit 620 through a user input interface 660 that is coupled to the system bus, but may be connected by other interface and bus structures, such as a parallel port, game port or a universal serial bus (USB). A monitor 691 or other type of display device is also connected to the system bus 621 via an interface, such as a graphics controller 690. In addition to the monitor, computers may also include other peripheral output devices such as speakers 697 and printer 696, which may be connected through an output peripheral interface 695.

The computer 610 may operate in a networked environment using logical connections to one or more remote computers, such as a remote computer 680. The remote computer 680 may be a personal computer, a server, a router, a network PC, a peer device or other common network node, and typically includes many or all of the elements described above relative to the computer 610, although only a memory storage device 681 has been illustrated in FIG. 22. The logical connections depicted in FIG. 22 include a local area network (LAN) 671 and a wide area network (WAN) 673, but may also include other networks. Such networking environments are commonplace in offices, enterprise-wide computer networks, intranets and the Internet.

When used in a LAN networking environment, the computer 610 is connected to the LAN 671 through a network interface or adapter 670. When used in a WAN networking environment, the computer 610 typically includes a modem 672 or other means for establishing communications over the WAN 673, such as the Internet. The modem 672, which may be internal or external, may be connected to the system bus 621 via the user input interface 660, or other appropriate mechanism. In a networked environment, program modules depicted relative to the computer 610, or portions thereof, may be stored in the remote memory storage device. By way of example, and not limitation, FIG. 22 illustrates remote application programs 685 as residing on memory device 681.

The communications connections 670 and 672 allow the device to communicate with other devices. The communications connections 670 and 672 are an example of communication media. The communication media typically embodies computer readable instructions, data structures, program modules or other data in a modulated data signal such as a carrier wave or other transport mechanism and includes any information delivery media. A “modulated data signal” may be a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Computer readable media may include both storage media and communication media.

In accordance with embodiments of the present invention, the components, process steps, and/or data structures may be implemented using various types of operating systems, computing platforms, computer programs, and/or general purpose machines. In addition, after having the benefit of this disclosure, those of ordinary skill in the art will recognize that devices of a less general purpose nature, such as hardwired devices, field programmable gate arrays (FPGAs), application specific integrated circuits (ASICs), or the like, may also be used without departing from the scope and spirit of the inventive concepts disclosed herein.

The computer program, according to an embodiment of the present invention, is a computerized system that requires the performance of one or more steps to be performed on or in association with a computerized device, such as, but not limited to, a server, a computer (i.e., desktop computer, laptop computer, netbook, or any machine having a processor), a dumb terminal that provides an interface with a computer or server, a personal digital assistant, mobile communications device, such as an cell phone, smart phone, or other similar device that provides computer or quasi-computer functionality, a mobile reader, such as an electronic document viewer, which provides reader functionality that may be enabled, through either internal components or connecting to an external computer, server, or global communications network (such as the Internet), to take direction from or engage in processes which are then delivered to the mobile reader. It should be readily apparent to those of skill in the art, after reviewing the materials disclosed herein, that other types of devices, individually or in conjunction with an overarching architecture, associated with an internal or external system, may be utilized to provide the “computerized” environment necessary for the at least one process step to be carried out in a machine/system/digital environment. It should be noted that the method aspects of the present invention are preferably computer-implemented methods and, more particularly, at least one step is preferably carried out using a computerized device.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

What is claimed is:

1. A system for assessing a personality profile comprising:
at least one database to hold known control data that includes correlations between musical preference patterns and psychological indications;
at least one processor configured to execute an input module, a processing module, and an output module;
wherein the input module receives information relating to at least one musical preference of an assessment subject, the information including a history of media selections that is stored in a memory and accessible using a user interface, and a response to a questionnaire;
wherein the processing module analyzes the information relating to the history of media selections and the response to the questionnaire by comparing the at least one musical preference to the musical preference patterns in the database; and
wherein the output module provides a personality profile of the assessment subject based on the analysis.

2. A system according to claim 1 wherein the at least one musical preference comprises at least one of a music playlist selection, a downloaded apps selection, a browser history selection, a downloaded images selection, a downloaded videos selection, a television recordings selection, and a browser cookie selection.

3. A system according to claim 2 wherein the at least one musical preference comprises a music playlist selection; and wherein the processing module is adapted to assign a score to the music playlist selection based on one or more of thematic content, lyrical content, artist characteristics, and musical content.

4. A system according to claim 3 wherein the processing module is adapted to determine a playlist scale score and an overall playlist score.

5. A system according to claim 2 wherein the processing module is adapted to determine an aggregate scale score and an overall aggregate score.

6. A system according to claim 1 wherein the processing module is adapted to assign a score to the questionnaire response based on one or more of a media preference, a media selection process, a demographic factor, and a functioning history factor.

7. A system according to claim 6 wherein the processing module is adapted to determine a questionnaire scale score and a total questionnaire score.

8. A system according to claim 1 wherein the questionnaire is transmittable over a network.

9. A system according to claim 1 wherein the computer-implemented interface is configured to execute on a device selected from the group consisting of a smartphone, a portable music player, a portable computer, and a digital video recorder.

10. A system according to claim 1 wherein the output module is adapted to generate at least one of a personality profile and a mental health diagnosis.

11. A system according to claim 10 wherein the personality profile includes at least one of a total score and a personality profile scale.

12. A system according to claim 10, wherein the mental health diagnosis includes a finding of psychological indications comprising at least one of a mental health issue, a mental illness, a mental disorder, and a mental health predisposition.

13. A computer-implemented method for assessing a personality profile, the method comprising:
receiving information relating to a history of media selections that includes at least one musical preference of an assessment subject, the information being stored in a memory and accessible using a user interface;
receiving a response to a questionnaire that includes data relating to the at least one musical preference of the assessment subject;
accessing a database that holds control data which includes correlations between musical preference patterns and psychological indications;
analyzing the information relating to the history of media selections and the response to the questionnaire by comparing the at least one musical preference to the musical preference patterns in the control data; and
providing a personality profile of the assessment subject based on the analysis, the personality profile being accessible using the user interface.

14. A method according to claim 13 wherein the at least one musical preference comprises at least one of a music playlist selection, a downloaded apps selection, a browser history selection, a downloaded images selection, a downloaded videos selection, a television recordings selection, and a browser cookie selection.

15. A method according to claim 13 wherein the at least one musical preference comprises a music playlist selection, and wherein analyzing the information further comprises assigning a score to the music playlist selection based on one or more of thematic content, lyrical content, artist characteristics, and musical content.

16. A method according to claim 15 wherein assigning the score to the music playlist selection further comprises determining a playlist scale score and an overall playlist score.

17. A method according to claim 15 wherein assigning the score to the music playlist selection further comprises determining an aggregate scale score and an overall aggregate score.

18. A method according to claim 13 further comprising assigning a score to the response to the questionnaire based on at least one of a media preference, a media selection process, a demographic factor, and a functioning history factor.

19. A method according to claim 18 wherein assigning the score to the response to the questionnaire further comprises determining a questionnaire scale score and a total questionnaire score.

20. A method according to claim 13 wherein the questionnaire is transmittable over a network.

21. A method according to claim 13 wherein the computer-implemented interface executes on a device selected from the group consisting of a smartphone, a portable music player, a portable computer, and a digital video recorder.

22. A method according to claim 13 wherein providing a personality profile of the assessment subject further comprises generating at least one of a personality profile and a mental health diagnosis.

23. A method according to claim 22 wherein the personality profile includes at least one of a total score and a personality profile scale.

24. A method according to claim 22 wherein the mental health diagnosis includes a finding of psychological indications comprising at least one of a mental health issue, a mental illness, a mental disorder, and a mental health predisposition.
